(19) United States
(12) Patent Application Publication
(10) Pub. No.: US 2011/0171124 A1
(43) Pub. Date: Jul. 14, 2011

(54) In situ methods for monitoring the EMT status of tumor cells in vivo

(75) Inventors: Joseph E. Bugaj, Longmont, CO (US); Robert C. Wild, Lafayette, CO (US)

(73) Assignee: OSI Pharmaceuticals, Inc.

(21) Appl. No.: 12/660,310

(22) Filed: Feb. 24, 2010

Related U.S. Application Data
(60) Provisional application No. 61/208,679, filed on Feb. 26, 2009.

(51) Int. Cl. A61K 51/06; A61K 49/00; G01N 33/00; A61P 35/00

(52) U.S. Cl. 424/1.49; 424/9.1; 800/3; 424/9.2

(57) ABSTRACT

The invention provides methods of determining in situ the EMT status of the cells of a tumor in a patient in vivo, including: (a) providing an EMT-status-detecting conjugate containing an antibody that binds to an EMT-status biomarker, and a reporter molecule; (b) introducing the conjugate into a patient with a tumor, such that the conjugate contacts the EMT-status biomarker of the cells of the tumor; (c) employing a means for detection of the signal from the conjugate at the tumor site; and (d) using a means for image analysis to localize and quantify the signal from the conjugate at the tumor site, a positive signal indicating the presence of epithelial tumor cells if the EMT-status biomarker is an epithelial biomarker, and mesenchymal tumor cells if the EMT-status biomarker is a mesenchymal biomarker. Such methods are useful for diagnosing patients who might benefit from treatment with drugs such as EGFR or IGF-1R kinase inhibitors, and for identifying and testing agents that inhibit tumor cells from undergoing an epithelial to mesenchymal transition. This invention also provides the above methods where an AFFIBODY® molecule that binds to an EMT-status biomarker is used instead of an antibody.

### CROSS-REFERENCE TO RELATED APPLICATIONS

This application claims the benefit of U.S. Provisional Application No. 61/208,679, filed Feb. 26, 2009, which is herein incorporated by reference in its entirety.

### BACKGROUND OF THE INVENTION

Cancer is a generic name for a wide range of cellular malignancies characterized by unregulated growth, lack of differentiation, and the ability to invade local tissues and metastasize, the latter involving epithelial to mesenchymal transition (EMT) of tumor cells. These neoplastic malignancies affect, with various degrees of prevalence, every tissue and organ in the body. The present invention is directed to new in situ diagnostic methods for accurately assessing and monitoring tumor EMT status in vivo, either in human patients or in experimental animal models of EMT used in pharmaceutical research. These methods will be useful for determining the EMT status of tumors prior to treatment with anti-cancer agents whose efficacy is affected by EMT (e.g. EGFR or IGF-1R kinase inhibitors). They will also be useful in the identification, testing and monitoring of new anti-cancer agents that inhibit EMT in tumor cells, and can be used in treating cancer patients, particularly in combination with other agents such as EGFR or IGF-1R kinase inhibitors, that have been reported to be less effective at inhibiting tumor cells that have undergone an EMT.

The epidermal growth factor receptor (EGFR) family comprises four closely related receptors (HER1/EGFR, HER2, HER3 and HER4) involved in cellular responses such as differentiation and proliferation. Over-expression of the EGFR kinase, or its ligand TGF-alpha, is frequently associated with many cancers, including breast, lung, colorectal, ovarian, renal cell, bladder, head and neck cancers, glioblastomas, and astrocytomas, and is believed to contribute to the malignant growth of these tumors. A specific deletion-mutation in the EGFR gene (EGFRvIII) has also been found to increase cellular tumorigenicity. Activation of EGFR stimulated signaling pathways promote multiple processes that are potentially cancer-promoting, e.g. proliferation, angiogenesis, cell motility and invasion, decreased apoptosis and induction of drug resistance. Increased HER1/EGFR expression is frequently linked to advanced disease, metastases and poor prognosis. For example, in NSCLC and gastric cancer, increased HER1/EGFR expression has been shown to correlate with a high metastatic rate, poor tumor differentiation and increased tumor proliferation.

Erlotinib (e.g. erlotinib HCl, also known as TARCEVA® or OSI-774) is an orally available inhibitor of EGFR kinase. In vitro, erlotinib has demonstrated substantial inhibitory activity against EGFR kinase in many human tumor cell lines. In a phase III trial, erlotinib monotherapy significantly prolonged survival, delayed disease progression and delayed worsening of lung cancer-related symptoms in patients with advanced, treatment-refractory NSCLC (Shepherd, F. et al. (2005) N. Engl. J. Med. 353(2):123-132). In November 2004 the U.S. Food and Drug Administration (FDA) approved TARCEVA® for the treatment of patients with locally advanced or metastatic non-small cell lung cancer (NSCLC) after failure of at least one prior chemotherapy regimen.

The development for use as anti-tumor agents of compounds that directly inhibit the kinase activity of IGF-1R, as well as antibodies that reduce IGF-1R kinase activity by blocking IGF-1R activation or antisense oligonucleotides that block IGF-1R expression, are also areas of intense research effort (e.g. see Larsson, O. et al (2005) Brit. J. Cancer 92:2097-2101; Ibrahim, Y. H. and Yee, D. (2005) Clin. Cancer Res. 11:944s-950s; Mitsiades, C. S. et al. (2004) Cancer Cell 5:221-230; Camirand, A. et al. (2005) Breast Cancer Research 7:R570—R579 (DOI 10.1186/bcr1028); Camirand, A. and Pollak, M. (2004) Brit. J. Cancer 90:1825-1829; Garcia-Echeverria, C. et al. (2004) Cancer Cell 5:231-239).

IGF-1R is a transmembrane RTK that binds primarily to IGF-1 but also to IGF-II and insulin with lower affinity. Binding of IGF-1 to its receptor results in activation of receptor tyrosine kinase activity, intermolecular receptor autophosphorylation and phosphorylation of cellular substrates (major substrates are IRS1 and Shc). The ligand-activated IGF-1R induces mitogenic activity in normal cells and plays an important role in abnormal growth. A major physiological role of the IGF-1 system is the promotion of normal growth and regeneration. Overexpressed IGF-1R (type 1 insulin-like growth factor receptor) can initiate mitogenesis and promote ligand-dependent neoplastic transformation. Furthermore, IGF-1R plays an important role in the establishment and maintenance of the malignant phenotype. Unlike the epidermal growth factor (EGF) receptor, no mutant oncogenic forms of the IGF-1R have been identified. However, several oncogenes have been demonstrated to affect IGF-1 and IGF-1R expression. The correlation between a reduction of IGF-1R expression and resistance to transformation has been seen. Exposure of cells to the mRNA antisense to IGF-1R RNA prevents soft agar growth of several human tumor cell lines. IGF-1R abrogates progression into apoptosis, both in vivo and in vitro. It has also been shown that a decrease in the level of IGF-1R below wild-type levels causes apoptosis of tumor cells in vivo. The ability of IGF-1R disruption to cause apoptosis appears to be diminished in normal, non-tumorigenic cells.

The IGF-1 pathway in human tumor development has an important role. IGF-1R overexpression is frequently found in various tumors (breast, colon, lung, sarcoma) and is often associated with an aggressive phenotype. High circulating IGF1 concentrations are strongly correlated with prostate, lung and breast cancer risk. Furthermore, IGF-1R is required for establishment and maintenance of the transformed phenotype in vitro and in vivo (Baserga R. Exp. Cell. Res., 1999, 253, 1-6). The kinase activity of IGF-1R is essential for the transforming activity of several oncogenes: EGFR, PDGFR, SV40 T antigen, activated Ras, Raf, and v-Src. The expression of IGF-1R in normal fibroblasts induces neoplastic phenotypes, which can then form tumors in vivo. IGF-1R expression plays an important role in anchorage-independent growth. IGF-1R has also been shown to protect cells from chemotherapy-, radiation-, and cytokine-induced apoptosis. Conversely, inhibition of endogenous IGF-1R by dominant negative IGF-1R, triple helix formation or antisense expression vector has been shown to repress transforming activity in vitro and tumor growth in animal models.

During most cancer metastases, an important change occurs in a tumor cell known as the epithelial-mesenchymal transition (EMT) (Thiery, J. P. (2002) Nat. Rev. Cancer 2:442-454; Savagner, P. (2001) Bioessays 23:912-923; Kang Y. and Massague, J. (2004) Cell 118:277-279; Julien-Grille, S., et al. Cancer Research 63:2172-2178; Bates, R. C. et al. (2003) Current Biology 13:1721-1727; Lu Z., et al. (2003) Cancer Cell. 4(6):499-515)). EMT does not occur in healthy cells except during embryogenesis. Epithelial cells, which are bound together tightly and exhibit polarity, give rise to mesenchymal cells, which are held together more loosely, exhibit a loss of polarity, and have the ability to travel. These mesenchymal cells can spread into tissues surrounding the original tumor, as well as separate from the tumor, invade blood and lymph vessels, and travel to new locations where they divide and form additional tumors. Recent research has demonstrated that epithelial cells respond well to EGFR and IGF-1R kinase inhibitors, but that after an EMT the resulting mesenchymal-like cells are much less sensitive to such inhibitors. (e.g. Thompson, S. et al. (2005) Cancer Res. 65(20):9455-9462; U.S. Patent Application 60/997,514). There is also a pressing need for anti-cancer agents that can prevent or reverse tumor cell EMT events (e.g. stimulate a mesenchymal to epithelial transition (MET)), or inhibit the growth of the mesenchymal-like tumor cells resulting from EMT. Such agents should be particularly useful when used in conjunction with other anti-cancer drugs such as EGFR and IGF-1R kinase inhibitors. In view of this, there is thus a need for improved methods for accurately assessing and monitoring tumor EMT status in vivo, for use both in experimental animals and in human patients, both for predicting the likely efficacy of anti-cancer agents whose actions are affected by tumor EMT status, and for testing the effects of new EMT inhibiting agents. The invention described herein provides new in situ methods for assessing tumor EMT status in vivo.

### SUMMARY OF THE INVENTION

The present invention provides a method of determining in situ the EMT status of the cells of a tumor in a patient, comprising: (a) providing an EMT-status-detecting conjugate comprising an antibody that binds to an EMT-status biomarker, and a reporter molecule that produces a detectable signal; (b) introducing said conjugate into a patient with a tumor, such that the conjugate contacts the EMT-status biomarker of the cells of the tumor; (c) employing a means for detection of the signal from the conjugate at the tumor site; and (d) using a means for image analysis to localize and quantify the signal from the conjugate at the tumor site, a positive signal indicating the presence of epithelial tumor cells if the EMT-status biomarker is an epithelial biomarker, and mesenchymal tumor cells if the EMT-status biomarker is a mesenchymal biomarker. This invention also provides methods of identifying agents that inhibit the cells of a tumor in a patient from undergoing an epithelial to mesenchymal transition by using the above method to determine which compounds have such activity in vivo, either in experimental animal models or in human patients.

This invention also provides a method for treating tumors in a patient with cancer, comprising: (a) assessing in situ the EMT status of the tumor cells by a method of the instant invention, and (b) administering to said patient a therapeutically effective amount of an EGFR kinase inhibitor. This invention also provides a method for treating tumors in a patient with cancer, comprising: (a) assessing in situ the EMT status of the tumor cells by a method of the instant invention, and (b) administering to said patient a therapeutically effective amount of an IGF-1R kinase inhibitor.

This invention also provides a composition comprising an EMT-status-detecting conjugate comprising an antibody that binds to an extracellular domain of an EMT-status biomarker and a reporter molecule that produces a detectable signal, for use in a method of determining in situ the EMT status of the cells of a tumor in a patient. In one embodiment the EMT-status biomarker is E-cadherin. In another embodiment the reporter molecule comprises Alexa Fluor 680 (AF680) fluorescent dye.

This invention also provides the above methods or compositions wherein an AFFIBODY® molecule that binds to an EMT-status biomarker is used instead of an antibody that binds to an EMT-status biomarker.

### BRIEF DESCRIPTION OF THE FIGURES

The file of this patent contains at least one drawing executed in color. Copies of this patent with color drawing(s) will be provided by the Patent and Trademark Office upon request and payment of the necessary fee.

FIG. 1: Top panel shows H358 tumor bearing mice (3) injected with AF680 E-cadherin NIR probe; Bottom panel shows same tumor bearing mice (3) injected with AF680 non-specific IgG.

FIG. 2: Histogram of Total Efficiency demonstrating 2.5 fold increase in uptake for the E-cadherin NIR probe compared to the non-specific probe.

FIG. 3: Dose titration of AF680 E-cadherin NIR probe in H358 tumor bearing mice injected with 40 μg, 20 μg and 10 μg respectively of the probe.

FIG. 4: Histogram demonstrating uptake of the E-cadherin NIR probe as a function of dose.

FIG. 5: A-F: PK study in H358 tumor bearing mice (n=3) injected with 20 ug of AF680 E-cadherin NIR probe imaged at 75 minutes (A), 6 hours (B), 27 hours (C), 48 hours (D), 120 hours (E) and 192 hours (F) post injection of probe.

FIG. 6: Graph of PK study showing uptake and clearance of AF680 E-cadherin NIR probe as a function of time (in hours post injection).

FIG. 7: Photographic image (A) of mice implanted bilaterally with an H358 xenograft (left flank) and a MDA-MB-231 xenograft (right flank). Specific uptake of the AF680 E-cadherin NIR probe in the H358 xenograft is shown in the fluorescent image (B).

FIG. 8: BxPC-3 tumor bearing mice injected with AF680 Non-specific IgG (top panel) and Af680 E-cadherin NIR probe (bottom panel) and imaged 48 hours post injection.

FIG. 9: Ex vivo images of excised tissues from BxPC-3 mouse study. Left section depicts tissues (n=4) from mice injected with the non-specific IgG probe, right section show tissues (n=4) from mice injected with the E-cadherin NIR probe.

FIG. 10: Histogram of AF680 NIR probe and non-specific IgG probe (NS) in excised tissues from BxPC-3 tumor bearing mice demonstrating greater than 3-fold increase in uptake of the E-cadherin NIR probe.

FIG. 11: Fluorescent images of H358 Tet-ON Zeb LV 195 xenografts in mice receiving no doxycycline treatment (top panel) compared to mice receiving 0.5 mg/mL doxycycline (bottom panel) in the drinking water for 7 days. Images depict in vivo change in E-cadherin expression on the tumors resulting from Zeb induction via the doxycycline treatment repressing E-cadherin expression in these tumors.

FIG. 12: Histogram from H359 Tet-ON Zeb mouse study showing ˜45% decrease in signal intensity of the E-cadherin NIR probe in tumors from mice receiving doxycycline treatment compared to mice receiving no doxycyline treatment.

FIG. 13: A schematic illustration of an NIR imaging device, including a 150 W halogen light source, a 610-650 nm bandpass filter, a cooled CCD, a 700 nm longpass filter and a computer with imaging software.

FIG. 14: A schematic illustration of an NIR imaging device, including a 737 nm/1.5 W laser, a laser light expander, a CCD camera controlled via a camera controller by a computer with imaging software, a mercury lamp, a mercury lamp light expander, and appropriate filters, as well as a light tight black box to house the subject.

### DETAILED DESCRIPTION OF THE INVENTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention is related. Still, certain elements are defined below for the sake of clarity and ease of reference, or for the purpose of the invention as described herein.

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

“Cell growth”, as used herein, for example in the context of “tumor cell growth”, unless otherwise indicated, is used as commonly used in oncology, where the term is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

“Tumor growth” or “tumor metastases growth”, as used herein, unless otherwise indicated, is used as commonly used in oncology, where the term is principally associated with an increased mass or volume of the tumor or tumor metastases, primarily as a result of tumor cell growth.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient with cancer. The term “treatment” as used herein, unless otherwise indicated, refers to the act of treating.

The phrase “a method of treating” or its equivalent, when applied to, for example, cancer refers to a procedure or course of action that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action.

As used herein, the term “patient” can refer to a human in need of treatment with an anticancer agent, and more preferably a human in need of such a treatment to treat cancer or a tumor, and who has one or more tumors. However, the term “patient” can also refer to non-human animals, preferably mammals such as dogs, cats, horses, cows, pigs, sheep and non-human primates, among others, that have one or more tumors, and are in need of treatment with an anticancer agent. The term “patient” can also refer to an experimental animal model for use in pharmaceutical research, which harbors one or more tumors, and can be used, for example, for evaluating the efficacy of an anti-cancer agent, wherein the tumor(s) can either be spontaneous or implanted (e.g. a mouse xenograft model, with human or other non-human animal tumor cells). The cancer or tumor type may be any of those listed herein below.

The term “therapeutically effective agent” means a composition that will elicit the biological or medical response of a tissue, system, animal or human that is being sought by the researcher, veterinarian, medical doctor or other clinician.

The term “therapeutically effective amount” or “effective amount” means the amount of the subject compound or combination that will elicit the biological or medical response of a tissue, system, animal or human that is being sought by the researcher, veterinarian, medical doctor or other clinician.

The term “antibody molecule” as used herein refers to a protein of the immunoglobulin (Ig) superfamily that binds noncovalently to certain substances (e.g. antigens and immunogens) to form an antibody-antigen complex, including but not limited to antibodies produced by hybridoma cell lines, by immunization to elicit a polyclonal antibody response, by chemical synthesis, and by recombinant host cells that have been transformed with an expression vector that encodes the antibody. In humans, the immunoglobulin antibodies are classified as IgA, IgD, IgE, IgG, and IgM and members of each class are said to have the same isotype. Human IgA and IgG isotypes are further subdivided into subtypes IgA₁, and IgA₂, and IgG_{I}, IgG₂, IgG₃, and IgG₄. Mice have generally the same isotypes as humans, but the IgG isotype is subdivided into IgG₁, IgG_{2b}, and IgG₃ subtypes. Thus, it will be understood that the term “antibody molecule” as used herein includes within its scope (a) any of the various classes or sub-classes of immunoglobulin, e.g., IgG, IgM, IgE derived from any of the animals conventionally used and (b) polyclonal and monoclonal antibodies, such as murine, chimeric, or humanized antibodies. Antibody molecules have regions of amino acid sequences that can act as an antigenic determinant, e.g. the Fc region, the kappa light chain, the lambda light chain, the hinge region, etc. An antibody that is generated against a selected region is designated anti-[region], e.g. anti-Fc, anti-kappa light chain, anti-lambda light chain, etc. An antibody is typically generated against an antigen by immunizing an organism with a macromolecule to initiate lymphocyte activation to express the immunoglobulin protein. The term antibody molecule, as used herein, also covers any polypeptide or protein having a binding domain that is, or is homologous to, an antibody binding domain, including, without limitation, single-chain Fv molecules (scFv), wherein a VH domain and a VL domain are linked by a peptide linker that allows the two domains to associate to form an antigen binding site (Bird et al., Science 242, 423 (1988) and Huston et al., Proc. Natl. Acad. Sci. USA 85, 5879 (1988)). These can be derived from natural sources, or they may be partly or wholly synthetically produced.

The term “antibody fragments” as used herein refers to fragments of antibody molecules that retain the principal selective binding characteristics of the whole antibody molecule. Particular fragments are well-known in the art, for example, Fab, Fab′, and F(ab′)₂, which are obtained by digestion with various proteases and which lack the Fc fragment of an intact antibody or the so-called “half-molecule” fragments obtained by reductive cleavage of the disulfide bonds connecting the heavy chain components in the intact antibody. Such fragments also include isolated fragments consisting of the light-chain-variable region, “Fv” fragments consisting of the variable regions of the heavy and light chains, and recombinant single chain polypeptide molecules in which light and heavy variable regions are connected by a peptide linker. Other examples of binding fragments include (i) the Fd fragment, consisting of the VH and CH1 domains; (ii) the dAb fragment (Ward, et al., Nature 341, 544 (1989)), which consists of a VH domain; (iii) isolated CDR regions; and (iv) single-chain Fv molecules (scFv) described above. In addition, arbitrary fragments can be made using recombinant technology that retains antigen-recognition characteristics.

The term “EMT-status-detecting conjugate” refers to a probe or complex comprising an antibody and a reporter molecule that produces a detectable signal, wherein the antibody is capable of binding to an extracellular domain of an EMT-status biomarker, and the reporter molecule is bound to the antibody covalently or non-covalently. However, preferably the antibody and the reporter molecule are bound covalently.

The term “EMT-status biomarker” as used herein refers to a biomarker protein, the expression level of which is characteristic of a cell's phenotype as being either epithelial or mesenchymal, in which at least part of their structure is expressed on the outside surface of a cell (i.e. an extracellular domain), and is thus accessible to an anti-biomarker antibody in vivo, and can thus be used to monitor in situ the EMT status of cells of a tumor during epithelial to mesenchymal transition (EMT). An example of such a biomarker is the epithelial biomarker E-cadherin. Additional examples of EMT-status biomarkers that may be useful in the context of the instant invention are disclosed herein below. The “EMT-status biomarker” protein may be human or non-human (e.g. mouse, rabbit, monkey, dog, etc.), depending on the source of the cells of the tumor. The term “target EMT-status antigen” or “target EMT-status biomarker” as used herein indicates the molecule of interest for the imaging methods described herein using EMT-status-detecting conjugates.

The term “reporter molecule” as used herein refers to a molecule or molecular complex that produces a detectable signal that can be used to label an antibody to produce an antibody-reporter conjugate, such that the signal can be used to locate and quantify a target EMT-status antigen or biomarker bound by the conjugate (i.e. an EMT-status-detecting conjugate) in a tumor of a patient in vivo. Examples of “reporter molecules” include NIR reporter molecules or a radionuclides, which emit or may be caused to emit a detectable electromagnetic signal or radiation.

The term “detectable signal” as used herein refers to a change in or an occurrence of, a signal from a reporter molecule that is directly or indirectly detectable either by observation or by instrumentation and the presence or magnitude of which is a function of the presence of a reporter-labeled target in a test subject (e.g. a tumor EMT-status biomarker labeled with an EMT-status-detecting conjugate). Typically, the detectable response is an optical response resulting in a change in the wavelength distribution patterns or intensity of absorbance or fluorescence, or a change in light scatter, fluorescence quantum yield, fluorescence lifetime, fluorescence polarization, a shift in excitation or emission wavelength or a combination of the above parameters, or a change in intensity of the products of radioactive decay from a radionuclide reporter, e.g. gamma-ray or x-ray photons. For optical responses, the detectable change in a given spectral property is generally an increase or a decrease. However, spectral changes that result in an enhancement of fluorescence intensity and/or a shift in the wavelength of fluorescence emission or excitation are also useful.

The term “fluorophore” as used herein refers to a composition that is inherently fluorescent. Fluorophores may be substituted to alter the solubility, spectral properties or physical properties of the fluorophore. Numerous fluorophores are known to those skilled in the art and include, but are not limited to coumarin, acridine, furan, dansyl, cyanine, pyrene, naphthalene, benzofurans, quinolines, quinazolinones, indoles, benzazoles, borapolyazaindacenes, oxazine and xanthenes, with the latter including fluoresceins, rhodamines, rosamine and rhodols as well as other fluorophores described in Richard P. Haugland, Molecular Probes Handbook Of Fluorescent Probes And Research Products (9th edition, Publisher: Molecular Probes, Inc., (Eugene, Oreg.), ISBN: 0971063605, including the CD-ROM, September 2002). As used herein fluorophores of the present invention are compatible with in vivo imaging, optically excited in tissue, and generally have an excitation wavelength of about 580 nm to about 800 nm or longer.

The term “illuminating” as used herein refers to the application of any energy or light source, particularly near-infrared (NIR) and visible light, capable of exciting the NIR dye EMT-status-detecting conjugates of the invention.

The term “means for detection” as used herein refers to a means of detecting a signal produced by a reporter-labelled conjugate localized in a tumor of a patient, and includes for example: systems or instruments for illuminating a patient and detecting any resulting fluorescent signal; radiation detectors such as PET scanner detector rings, and gamma cameras and counters. Such “means for detection” are well known by those of skill in the art of in vivo imaging, and typically convert the detected signal into an electrical signal representative of the detected signal. Note that the term as used herein refers to systems, instruments or detectors that detect a signal spontaneously emanating from a reporter molecule (e.g. a radionuclide) as well as to those that detect a signal that they cause to be emitted, for example by illuminating a subject containing a reporter such as fluorophore.

The term “in vivo imaging” as used herein refers to methods or processes in which the structural, functional, or physiological state of a patient is examinable without the need for biopsy or life-ending sacrifice.

The term “non-invasive in vivo imaging” as used herein refers to methods or processes in which the structural, functional, or physiological state of a patient is examinable by remote physical probing without the need for breaching the physical integrity of the outer (skin) or inner (accessible orifices) surfaces of the body, except in the initial delivery of a probe containing a reporter molecule, e.g. an EMT-status-detecting conjugate.

The term “means for image analysis” as used herein refers to instrument systems or technologies, typically computer controlled and comprising both hardware and software components, that can be utilized for localizing and quantifying a signal produced by a reporter-labelled conjugate in a tumor of a patient that is detected by “a detector means”. Such “means for image analysis” are well known by those of skill in the art of in vivo imaging, and typically produce a two- or three-dimensional map of the signal from the target site (i.e. tumor in the instant application), typically color coded to indicate the location and amount of signal detected. Examples of instruments capable of performing such image analysis include NIR imaging systems; instruments for performing positron emission tomography (PET), using for example localization of the positron annihilation event, or image reconstruction using coincidence statistics; and instruments for performing single photon emission computed tomography (SPECT).

The term “kit” as used refers to a packaged set of related components, typically one or more compounds or compositions, and optionally an instruction set for their use in a method or assay.

The term “microsphere or microparticle” as used herein refers to particles of a size typically measured in the range from about 0.01 to about 10 microns and composed of any organic or inorganic material whose chemical and physical properties allow formation of functionally stable particles in this size range, which are preferably amenable to staining or association with a NIR dye. Preferred microspheres are polymeric organic particles, and can be comprised of a block copolymer. Some preferred microspheres for use in optical imaging of disease states are describe in PCT/US2006/061792, filed on Dec. 8, 2006, the contents of which are incorporated by reference as if set forth fully herein. In particular, compositions described in the contrast reagent section of PCT/US2006/061792 are contemplated for use as conjugates described herein.

The term “near IR dye” or “near IR reporter molecule” or “NIR dye” or “NIR reporter molecule” as used herein indicates a dye or reporter molecule with an excitation wavelength of about 580 nm to about 800 nm. Preferably, the NIR dyes emit in the range of about 590 nm to about 860 nm. Most preferred NIR dyes are excited from about 680 to about 790 nm. Examples of dyes suitable for use in the instant invention include, Alexa Fluor 660 Dye, Alexa Fluor 680 dye (AF680), Alexa Fluor 700 dye, Alexa Fluor 750 dye, and Alexa Fluor 790 dye (Berlier J E, et al., J. Histochem. Cytochem. 2003 (51):12 1699-1712; Panchuk-Voloshina N, et al., J Histochem Cytochem 1999 (47):9 1179-1188). The NIR dyes are particularly advantageous for in vivo imaging because they can be selectively visualized without exciting endogenous materials present in a patient. Some of the NIR dyes have a large stokes shift, such that the excitation and emission wavelengths are separated by at least 20, 30, 40, 50, 60, 70 or 80 nm.

“Inducibly express”, as used herein, when referring for example to cells which have been engineered to “inducibly express” a protein, means that the protein expression is only turned on by the presence (or absence) of an inducing agent that controls transcription of the gene encoding the protein, which will preferably be incorporated into the cells by stable transformation with a construct containing the gene for the encoding protein under the control of a promoter that is responsive to the inducing agent (i.e. the gene encoding the protein is operably linked to a nucleotide sequence regulating the gene expression, which nucleotide sequence comprises a promoter sequence whose activity can be controlled by the presence of an inducing agent). One example of such an inducible promoter is a tetracycline (tet)-responsive promoter (e.g. a Tet-on system; e.g. see Gossen, M. et al. (1995) Science 268:1766-1769). Such inducible gene expression systems for controlling the expression levels of specific genes of interest are well known in the art (e.g. see Blau, H. M. and Rossi, F. M. V. (1999) Proc. Natl. Acad. Sci. USA 96:797-799; Yamamoto, A. et al. (2001) Neurobiology of Disease 8:923-932; Clackson, T. (2000) Gene Therapy 7:120-125).

The present invention derives from research that provided methods for determining which tumors will respond most effectively to treatment with protein-tyrosine kinase inhibitors (e.g. Thompson, S. et al. (2005) Cancer Res. 65(20):9455-9462; U.S. Patent Application 60/997,514) based on whether the tumor cells have undergone an epithelial to mesenchymal transition (“EMT”; Thiery, J. P. (2002) Nat. Rev. Cancer 2:442-454; Savagner, P. (2001) Bioessays 23:912-923; Kang Y. and Massague, J. (2004) Cell 118:277-279; Julien-Grille, S., et al. Cancer Research 63:2172-2178; Bates, R. C. et al. (2003) Current Biology 13:1721-1727; Lu Z., et al. (2003) Cancer Cell. 4(6):499-515). This research demonstrated that epithelial cells respond well to EGFR and IGF-1R kinase inhibitors, but that after an EMT the resulting mesenchymal-like cells are much less sensitive to such inhibitors. Biomarkers can be used to determine whether tumor cells have undergone an EMT (Thomson, S. et al. (2005) Cancer Res. 65(20):9455-9462). As a result of such work it became apparent that new therapeutic approaches would be required to find agents that were capable of inhibiting the genesis, growth and/or function of such mesenchymal-like cells, which are thought to be an important element in the invasive and metastatic properties of tumors.

A considerable body of work is emerging that is beginning to delineate the biochemical pathways involved in regulating tumor EMT events, and to characterize the resultant mesenchymal-like tumor cells. For example, experiments using specific siRNA inhibitors of the expression of various protein products produced by mesenchymal-like tumor cells have demonstrated that reduced expression of the products of certain genes can specifically inhibit the growth of mesenchymal-like tumor cells. Thus pharmacological agents that also specifically inhibit the expression of the protein products encoded by these genes, or specifically inhibit the biological activity of the expressed proteins (e.g. phosphotransferase activity), such as specific antibodies to expressed proteins that possess an extracellular domain, antisense molecules, ribozymes, or small molecule enzyme inhibitors (e.g. protein kinase inhibitors), are similarly expected to be agents that will also specifically inhibit the growth of mesenchymal-like tumor cells. The anti-tumor effects of a combination of an EGFR or IGF-1R kinase inhibitor with such an agent should be superior to the anti-tumor effects of these kinase inhibitors by themselves, since such a combination should effectively inhibit both epithelial and mesenchymal-like tumor cells, and thus co-administration of such agents with EGFR or IGF-1R kinase inhibitors should be effective for treatment of patients with advanced cancers such as NSCL, pancreatic, colon or breast cancers.

Given the identification of key targets for the discovery and development of agents that will inhibit the growth of mesenchymal-like tumor cells, or the EMT process, there is thus a pressing need for improved methods to evaluate agents identified by in vitro screening methods to determine if they have the predicted effect of inhibiting the formation, growth and/or migration of mesenchymal-like tumor cells in vivo, both in animal model systems during drug development, and in human patients when evaluating drug efficacy. There is also a need for improved diagnostic methods for determining the EMT status of cells in patients' tumors in order to predict which are likely to be susceptible to inhibition by EGFR or IGF-1R kinase inhibitors and thus good candidates for such treatments, and which would likely benefit from additional agents that inhibit EMT or the resulting mesenchymal tumor cells.

A major shortcoming of current methods for determining EMT status in tumors in vivo derives from the fact that human tumors are typically heterogenous in nature, often comprising multiple tumor cell phenotypes, including both epithelial and mesenchymal cells, and other cell types (e.g. normal tissue cells, stromal cells, vasculature cells) which can express epithelial and/or mesenchymal cell markers, and most of these cell types are typically not evenly distributed within the tumor. Mesenchymal cells for example are frequently associated primarily with the leading edge or invasive front of solid tumors (Lee, J. M. et al. (2006) J. Cell. Biol. 172(7): 973-981). Thus current methods for determining EMT status which rely on biomarker determination in small biopsy samples can produce misleading results due to sampling variations, particularly if only one biopsy is relied upon. Such methodological deficiencies will be exacerbated when one attempts to monitor the progess of drug treatment over a period of time using a series of biopsies. Such deficiencies could perhaps be partially ameliorated by using multiple biopsies to sample different areas of the tumor, but such a highly invasive approach would not be desirable in most instances, and may not be possible in many others.

The data presented in the Examples herein below demonstrate that surprisingly it is possible to determine the EMT status of cells in a tumor in situ, without the need for biopsy samples. Using an animal xenograft model implanted with human tumor cells for which the EMT status is known, and can be experimentally manipulated, the inventors have demonstrated that it is possible to determine the EMT status of human tumor cells of a tumor in situ. This was done by injecting the animal (a nude mouse) with an anti-EMT-cell-surface-biomarker antibody that was labeled with a reporter molecule (i.e. an “EMT-status-detecting conjugate”) from which a signal could be detected and measured, allowing one to locate and quantify the EMT biomarker (e.g. E-cadherin) on the tumor and thereby determine the EMT status of the tumor cells. The antibody was labeled with an NIR dye that could readily be detected by illuminating at the appropriate wavelength of the electromagnetic spectrum, monitoring its fluorescence at its emission spectrum, and quantified using imaging analysis technology. Alternative labelling methods are also contemplated that are more appropriate for application to larger animals (e.g. humans, monkeys), such as a positron-emitting radionuclides (i.e. radioactive tracers detectable by PET scans), or single photon emission computed tomography (SPECT) probes. Experiments using nude mice implanted with mouse tumor cells were similarly successful. It should be noted that the results reported herein are particularly surprising given that the antibodies used bind to human or mouse proteins, and that many tissues in the mouse express the EMT biomarker protein detected. Thus one might have predicted that the non-tumor background signal would have obscured any tumor signal produced, and thus prevented one from measuring a sufficiently large signal-to-noise in order to monitor tumor EMT status. The data described herein exemplifies new methods that can be used for identifying and studying pharmaceutical agents that inhibit EMT, and for the treatment of patients with such agents, or other agents whose efficacy appears to be dependent on tumor cell EMT status (e.g. EGFR kinase inhibitors).

Accordingly, the present invention provides a method of determining in situ the EMT status of the cells of a tumor in a patient, comprising: (a) providing an EMT-status-detecting conjugate comprising an antibody that binds to an EMT-status biomarker, and a reporter molecule that produces a detectable signal; (b) introducing said conjugate into a patient with a tumor, such that the conjugate contacts the EMT-status biomarker of the cells of the tumor; (c) employing a means for detection of the signal from the conjugate at the tumor site; and (d) using a means for image analysis to localize and quantify the signal from the conjugate at the tumor site, a positive signal indicating the presence of epithelial tumor cells if the EMT-status biomarker is an epithelial biomarker, and mesenchymal tumor cells if the EMT-status biomarker is a mesenchymal biomarker. In one embodiment of this method, non-invasive in vivo imaging is employed, wherein the means for detection of the signal from the conjugate at the tumor site is external to the patient. In another embodiment of this method wherein in vivo imaging is employed, the means for detection of the signal from the conjugate at the tumor site is attached to a physical probe that can penetrate accessible orifices of a patient.

In the methods of this invention the patient may be a human with cancer in need of treatment. Determination of the EMT status of the tumor cells of the patient can be used in determining the preferred method of treatment. Certain anti-cancer agents, such as for example EGFR or IGF-1R kinase inhibitors, have been demonstrated to be more effective at inhibiting epithelial cells than mesenchymal cells. Also, as agents which inhibit EMT become available, the methods of this invention will provide valuable diagnostic tools to identify patients who will most benefit from treatment with such agents.

Reference to an “antibody” in the methods or compositions of this invention optionally includes “antibody molecules”, “antibody fragments, or mixtures of such antibody molecules or fragments.

This invention also provides a method of identifying an agent that inhibits the cells of a tumor in a patient from undergoing an epithelial to mesenchymal transition, comprising: (a) introducing a test agent that into said patient such that the agent contacts the cells of the tumor; (b) contacting the patient with an agent capable of inducing the cells of the tumor to undergo EMT; (c) providing an EMT-status-detecting conjugate comprising an antibody that binds to an EMT-status biomarker, and a reporter molecule that produces a detectable signal; (d) introducing said conjugate into the patient, such that the conjugate contacts the EMT-status biomarker of the cells of the tumor; (e) employing a means for detection of the signal from the conjugate at the tumor site; (f) using a means for image analysis to localize and quantify the signal from the conjugate at the tumor site, a positive signal indicating the presence of epithelial tumor cells if the EMT-status biomarker is an epithelial biomarker, and mesenchymal tumor cells if the EMT-status biomarker is a mesenchymal biomarker, and (g) comparing the signal in (f) with that produced in a similarly treated patient but that was not treated with the test agent as in (a), thus identifying whether said test agent is an agent that can inhibit tumor cells from undergoing an epithelial to mesenchymal transition. In a preferred embodiment of this method the patient is an animal model of cancer (i.e. non-human), wherein agents are known that can induce EMT in the cells of the tumor(s) in the model. Several agents are listed herein that are known inducers of EMT. In a preferred animal model, the animal is an immune-deficient animal (e.g. an immune-deficient mouse, such as a nude mouse, also known as a Foxn1nu mouse). The latter can be used as a host for a tumor graft (i.e. tumor xenograft), using tumor cells from any other animal (e.g. rat, mouse, dog, monkey etc.), but preferably human tumor cells. In one embodiment the xenograft cells are tumor epithelial cells that have been engineered to inducibly express a protein that stimulates an epithelial to mesenchymal transition in the cells. The protein that is inducibly expressed and stimulates an epithelial to mesenchymal transition may for example be Snail, Zeb-1, or constitutively active TGF-beta. Suitable cells for this purpose include for example human NSCLC H358 cells (e.g. see U.S. patent application 61/068,612, which discloses how to make and use inducible H358 EMT models; Thomson, S. et al. (2008) Clin. Exp. Metastasis. 25(8):843-54).

Alternatively, the methods of this invention can be used for the identification of agents that inhibit cells of a tumor in a patient from undergoing an epithelial to mesenchymal transition induced by factors endogenous to the patient. Such factors may, for example, be autocrine, paracrine, or endocrine in nature. Thus, this invention also provides a method of identifying an agent that inhibits the cells of a tumor in a patient from undergoing an epithelial to mesenchymal transition, comprising: (a) introducing a test agent that into said patient such that the agent contacts the cells of the tumor; (b) providing an EMT-status-detecting conjugate comprising an antibody that binds to an EMT-status biomarker, and a reporter molecule that produces a detectable signal; (c) introducing said conjugate into the patient, such that the conjugate contacts the EMT-status biomarker of the cells of the tumor; (d) employing a means for detection of the signal from the conjugate at the tumor site; (e) using a means for image analysis to localize and quantify the signal from the conjugate at the tumor site, a positive signal indicating the presence of epithelial tumor cells if the EMT-status biomarker is an epithelial biomarker, and mesenchymal tumor cells if the EMT-status biomarker is a mesenchymal biomarker, and (f) comparing the signal in (e) with that produced in a similarly treated patient but that was not treated with the test agent as in (a), thus identifying whether said test agent is an agent that can inhibit tumor cells from undergoing an epithelial to mesenchymal transition. In one embodiment of this method the patient may be a human patient being treated to evaluate the effectiveness of an agent to inhibit cells of a tumor in a patient from undergoing an epithelial to mesenchymal transition, wherein the agent has been previously demonstrated to possess such activity in other human patients, animal models of cancer (e.g. human xenografts in mice), and/or in vitro EMT cellular models.

In the methods of the invention described herein for identifying an agent that inhibits the cells of a tumor in a patient from undergoing an epithelial to mesenchymal transition, when the EMT-status biomarker is an epithelial biomarker, a stronger positive signal in the tumor treated with the test agent relative to the control patient tumor (without the test agent) indicates that the agent is inhibiting EMT. When the EMT-status biomarker is a mesenchymal biomarker, a weaker positive signal in the tumor treated with the test agent relative to the control patient tumor (without the test agent) indicates that the agent is inhibiting EMT.

This invention also provides a method for treating tumors in a patient with cancer, comprising: (a) assessing in situ the EMT status of the tumor cells by a method of the instant invention, and (b) administering to said patient a therapeutically effective amount of an EGFR kinase inhibitor. In a preferred embodiment the EGFR kinase inhibitor is erlotinib. Other suitable EGFR inhibitors are listed herein below.

This invention thus provides a method for treating a tumor in a patient with cancer, comprising: determining in situ the EMT status of the cells of a tumor in a patient, by (a) providing an EMT-status-detecting conjugate comprising an antibody that binds to an EMT-status biomarker, and a reporter molecule that produces a detectable signal; (b) introducing said conjugate into a patient with a tumor, such that the conjugate contacts the EMT-status biomarker of the cells of the tumor; (c) employing a means for detection of the signal from the conjugate at the tumor site; and (d) using a means for image analysis to localize and quantify the signal from the conjugate at the tumor site, a positive signal indicating the presence of epithelial tumor cells if the EMT-status biomarker is an epithelial biomarker, and mesenchymal tumor cells if the EMT-status biomarker is a mesenchymal biomarker, and administering to said patient a therapeutically effective amount of an EGFR kinase inhibitor (e.g. erlotinib). In one embodiment of this method the EMT status of the tumor cells are determined to be epithelial, indicating that the growth of the tumor cells is likely to be sensitive to inhibition by an EGFR kinase inhibitor. In another embodiment of this method the EMT status of the tumor cells are determined to be principally epithelial, indicating that the growth of the majority of the tumor cells is likely to be sensitive to inhibition by an EGFR kinase inhibitor. In another embodiment of this method the EMT status of the tumor cells are determined to be a percentage epithelial selected from: at least 20%; at least 40%; at least 60%; or at least 80% epithelial, indicating that the growth of at least that percentage of tumor cells is likely to be sensitive to inhibition by an EGFR kinase inhibitor. In another embodiment of this method the EMT status of the tumor cells are determined to be mesenchymal or principally mesenchymal, indicating that the growth of the tumor cells is less likely to be sensitive to inhibition by an EGFR kinase inhibitor. However, in the latter embodiment a physician may still determine that administration of an EGFR kinase inhibitor is a potentially useful course of action depending, for example, on the condition of the patient, the past responses to other anticancer drugs, or the availability of other agents that may be used in combination with the EGFR kinase inhibitor to potentially enhance the effectiveness of the treatment.

This invention also provides a method for treating tumors in a patient with cancer, comprising: (a) assessing in situ the EMT status of the tumor cells by a method of the instant invention, and (b) administering to said patient a therapeutically effective amount of an IGF-1R kinase inhibitor. In a preferred embodiment the IGF-1R kinase inhibitor is OSI-906. Other suitable IGF-1R inhibitors are listed herein below.

This invention thus provides a method for treating a tumor in a patient with cancer, comprising: determining in situ the EMT status of the cells of a tumor in a patient, by (a) providing an EMT-status-detecting conjugate comprising an antibody that binds to an EMT-status biomarker, and a reporter molecule that produces a detectable signal; (b) introducing said conjugate into a patient with a tumor, such that the conjugate contacts the EMT-status biomarker of the cells of the tumor; (c) employing a means for detection of the signal from the conjugate at the tumor site; and (d) using a means for image analysis to localize and quantify the signal from the conjugate at the tumor site, a positive signal indicating the presence of epithelial tumor cells if the EMT-status biomarker is an epithelial biomarker, and mesenchymal tumor cells if the EMT-status biomarker is a mesenchymal biomarker, and administering to said patient a therapeutically effective amount of an IGF-1R kinase inhibitor (e.g. OSI-906). In one embodiment of this method the EMT status of the tumor cells are determined to be epithelial, indicating that the growth of the tumor cells is likely to be sensitive to inhibition by an IGF-1R kinase inhibitor. In another embodiment of this method the EMT status of the tumor cells are determined to be principally epithelial, indicating that the growth of the majority of the tumor cells is likely to be sensitive to inhibition by an IGF-1R kinase inhibitor. In another embodiment of this method the EMT status of the tumor cells are determined to be a percentage epithelial selected from: at least 20%; at least 40%; at least 60%; or at least 80% epithelial, indicating that the growth of at least that percentage of tumor cells is likely to be sensitive to inhibition by an IGF-1R kinase inhibitor. In another embodiment of this method the EMT status of the tumor cells are determined to be mesenchymal or principally mesenchymal, indicating that the growth of the tumor cells is less likely to be sensitive to inhibition by an IGF-1R kinase inhibitor. However, in the latter embodiment a physician may still determine that administration of an IGF-1R kinase inhibitor is a potentially useful course of action depending, for example, on the condition of the patient, the past responses to other anticancer drugs, or the availability of other agents that may be used in combination with the IGF-1R kinase inhibitor to potentially enhance the effectiveness of the treatment.

This invention also provides a composition comprising an EMT-status-detecting conjugate comprising an antibody that binds to an extracellular domain of an EMT-status biomarker and a reporter molecule that produces a detectable signal, for use in a method of determining in situ the EMT status of the cells of a tumor in a patient. In a preferred embodiment the antibody and the reporter molecule are covalently bound. The EMT-status biomarker may be an epithelial or a mesenchymal biomarker. Examples of suitable EMT-status biomarkers are listed herein. The reporter molecule may be for example an NIR dye or a radionuclide, of which several suitable examples are listed herein. In one embodiment the reporter molecule comprises Alexa Fluor 680 fluorescent dye.

This invention also provides a composition comprising an EMT-status-detecting conjugate comprising an antibody that binds to an extracellular domain of E-cadherin and a reporter molecule that produces a detectable signal, for use in a method of determining in situ the EMT status of the cells of a tumor in a patient. In a preferred embodiment the antibody and the reporter molecule are covalently bound. In one embodiment the reporter molecule comprises Alexa Fluor 680 fluorescent dye.

This invention also provides a method for in situ imaging an EMT-status biomarker in a tumor of a patient, wherein the method comprises; a) providing an EMT-status-detecting conjugate comprising an antibody that binds to an EMT-status biomarker and an NIR reporter molecule that produces a detectable signal; b) introducing the EMT-status-detecting conjugate into the patient to form a contacted patient; c) illuminating the contacted patient with an appropriate wavelength to form an illuminated patient; and d) observing the illuminated patient wherein the EMT-status biomarker is imaged; wherein the EMT-status biomarker indicates the stage of epithelial-to-mesenchymal transition of the patient's tumor cells.

This invention also provides a method for in situ imaging an EMT-status biomarker in a tumor of a patient, wherein the method comprises; a) providing an EMT-status-detecting conjugate comprising an antibody that binds to an EMT-status biomarker and an radionuclide reporter molecule that produces a detectable signal; b) introducing the EMT-status-detecting conjugate into the patient to form a contacted patient; and c) observing the contacted patient wherein the EMT-status biomarker is imaged; wherein the EMT-status biomarker indicates the stage of epithelial-to-mesenchymal transition of the patient's tumor cells.

This invention also provides a non-invasive method of detecting the EMT status of the cells of a tumor in vivo in a patient, the method comprising: administering a composition to the patient comprising an EMT-status-detecting conjugate comprising an antibody that binds to an EMT-status biomarker and a reporter molecule that produces a detectable signal, and obtaining an in vivo image of the detectable signal for at least part of the patient, in order to detect the EMT status of the cells of the tumor in vivo in the patient.

In one embodiment of any of the imaging methods of this invention, non-invasive in vivo imaging is employed, wherein the means for detection of the signal from the conjugate at the tumor site is external to the patient. In another embodiment of any of the in vivo imaging methods of this invention, the means for detection of the signal from the conjugate at the tumor site is attached to a physical probe that can penetrate accessible orifices of a patient (e.g. GI tract).

In any of the methods of the instant invention, as an additional control step, the method steps may be repeated using instead of an EMT-status-detecting conjugate, a conjugate comprising a non-specific antibody (e.g. that binds to an epitope that is not present in the patient) labeled to the same or similar degree with the same reporter molecule, and comparing the signals from each of the conjugates at the tumor site to determine the amount of signal from the EMT-status-detecting conjugate that is specific to the EMT biomarker. This additional control step may be used to establish the degree to which a signal observed with an EMT-status-detecting conjugate is EMT biomarker specific. Once a non-specific background signal level is established it may not be necessary to repeat this control step with every EMT status determination.

In preferred embodiments of the methods or compositions of this invention the reporter molecule of the EMT-status-detecting conjugate that produces a detectable signal comprises an NIR reporter molecule or a radionuclide, which emits or may be caused to emit a detectable electromagnetic signal or radiation. NIR reporter molecules are preferred where the patient is small, e.g. a mouse, rat, rabbit, small monkey. For larger animals (e.g. humans, other large primates, dogs) a radionuclide reporter is preferred due to the superior tissue penetration of the detectable signal from such a reporter. There are many examples of such reporter molecules known in the art that would be suitable for use in the invention described herein, some of which are described herein. The chosen reporter molecule should not hinder the target specific reporter conjugates ability to travel relatively freely within the circulating blood and lymph of a patient until their preferential sequestration occurs at a tumor site by the target EMT-status antigen.

Any fluorescent dye known to one of skill in the art having an excitation wavelength compatible with in vivo imaging can be used as a NIR reporter molecule for the above described EMT-status-detecting conjugates. Typically the fluorescent dyes will have an excitation wavelength of at least 580 nm. Suitable NIR reporter molecules include for example a fluorescent dye(s) having an excitation wavelength compatible with in vivo imaging, typically about 580 nm to about 800 nm. A wide variety of long wavelength fluorescent dyes that may be suitable for conjugation to proteins and peptides are already known in the art (e.g. Richard P. Haugland, Molecular Probes Handbook Of Fluorescent Probes And Research Products (2002) (Supra); Published International Application No. WO 2007/109809). Detection of probe fluorescence in the near infrared (NIR) range of the electromagnetic spectrum represents an especially promising in vivo imaging modality as body tissues tend to be relatively transparent in this range (B. C. Wilson, Optical properties of tissues. Encyclopedia of Human Biology, 1991, 5, 587-597).

A fluorescent dye or fluorophore of the present invention is any chemical moiety that exhibits an absorption maximum beyond 580 nm and that is optically excited and observable in tissue. Dyes of the present invention include, without limitation; a pyrene, an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a carbocyanine (including any corresponding compounds in U.S. Ser. Nos. 09/968,401; 09/969,853 and 11/150,596 and U.S. Pat. Nos. 6,403,807; 6,348,599; 5,486,616; 5,268,486; 5,569,587; 20 5,569,766; 5,627,027; 6,664,047; 6,048,982 AND 6,641,798), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 25 6,339,392; 5,451,343 and U.S. Ser. No. 09/922,333), an oxazine or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and 30 benzphenalenone (including any corresponding compounds disclosed in U.S. Pat. No. 4,812,409) and derivatives thereof. As used herein, oxazines include resorufins (including any corresponding compounds disclosed in 5,242,805), aminooxazinones, diaminooxazines, and their benzo-substituted analogs.

Where the dye is a xanthene, the dye is optionally a fluorescein, a rhodol (including any corresponding compounds disclosed in U.S. Pat. Nos. 5,227,487 and 5,442,045), a rosamine or a rhodamine (including any corresponding compounds in U.S. Pat. Nos. 5,798,276; 5,846,737; 5,847,162; 6,017,712; 6,025,505; 6,080,852; 6,716,979; 6,562,632). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171). Fluorinated xanthene dyes have been described previously as possessing particularly useful fluorescence properties (Int. Publ. No. WO 97/39064 and U.S. Pat. No. 6,162,931).

Thus, in one embodiment, the NIR dye is selected from the group consisting of a pyrene, an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a carbocyanine, a carbostyryl, a porphyrin, a salicylate, an anthranilate, an azulene, a perylene, a pyridine, a quinoline, a borapolyazaindacene, a xanthene, an oxazine, a benzoxazine, a resorufin, a carbazine, a phenalenone, a coumarin, a benzofuran, a benzphenalenone and derivatives thereof.

In one embodiment the dye has an emission spectrum with its maximum greater than about 600 nm. In a further embodiment the dye or fluorophore has an emission spectrum with its maximum greater than about 620 nm, an emission maximum greater than about 650 nm, an emission maximum great than about 700 nm, an emission maximum greater than about 750 nm, or an emission maximum greater than about 800 nm. In another embodiment, the NIR dye has an excitation wavelength of about 580 nm to about 800 nm. More particularly, the NIR dye has an excitation wavelength of about 660 nm to about 790 nm. In another embodiment, the NIR dye has an emission wavelength of about 600 nm to about 850 nm. In one aspect the dye is a cyanine dye. Preferred are those dyes sold under the trade name Alexa Fluor® dye or spectrally similar dyes sold under the trade names Cy® dyes, Atto dyes or Dy® dyes. Preferred Alexa Fluor dyes include, Alexa Fluor 660 dye, Alexa Fluor 680 dye, Alexa Fluor 700 dye, Alexa Fluor 750 dye, and Alexa Fluor 790 dye. Additional dyes that have been described and/or are commercially available, include Cy5.5 (Amersham, Arlington Heights, Ill.); NIR-1 (Dojindo, Kumamoto, Japan); IRD382 (LI-COR, Lincoln, Nebr.); La Jolla Blue (Diatron, Miami, Fla.); ICG (Alcorn, Lincolnshire, Ill.); and ICG derivatives (Serb Labs, Paris, France).

Typically the dye contains one or more aromatic or heteroaromatic rings, that are optionally substituted one or more times by a variety of substituents, including without limitation, halogen, nitro, sulfo, cyano, alkyl, perfluoroalkyl, alkoxy, alkenyl, alkynyl, cycloalkyl, arylalkyl, acyl, aryl or heteroaryl ring system, benzo, or other substituents typically present on chromophores or fluorophores known in the art.

The dye in the EMT-status-detecting conjugate can be directly bound through a single covalent bond, cross-linked or bound through a linker, such as a series of stable covalent bonds incorporating 1-20 nonhydrogen atoms selected from the group consisting of C, N, O, S and P that covalently attach the fluorescent dye to the antibody or other moiety such as a chemically reactive group or a biological and non-biological component. The conjugation or linker may involve a receptor binding motif, such as biotin/avidin.

In another embodiment, the antibodies can be conjugated with fluorescent or light scattering nanocrystals [Yguerabide, J. and Yguerabide, E E, 2001 J. Cell Biochem Suppl. 37: 71-81; U.S. Pat. Nos. 6,214,560; 6,586,193 and 6,714,299]. These fluorescent nanocrystals can be semiconductor nanocrystals or doped metal oxide nanocrystals. Nanocrystals typically are comprised of a core comprised of at least one of a Group II-VI semiconductor material (of which ZnS, and CdSe are illustrative examples), or a Group III-V semiconductor material (of which GaAs is an illustrative example), a Group IV semiconductor material, or a combination thereof. The core can be passivated with a semiconductor overlayering (“shell”) uniformly deposited thereon. For example, a Group II-VI semiconductor core may be passivated with a Group II-VI semiconductor shell (e.g., a ZnS or CdSe core may be passivated with a shell comprised of YZ wherein Y is Cd or Zn, and Z is S, or Se). Nanocrystals can be soluble in an aqueous-based environment. An attractive feature of semiconductor nanocrystals is that the spectral range of emission can be changed by varying the size of the semiconductor core.

After selection of an appropriate dye with the desired spectral characteristics, typically where the excitation wavelength is at least 580 nm, the dyes are conjugated to an anti-EMT-status biomarker antibody, using methods well known in the art (Haugland, Molecular Probes Handbook, supra, (2002)). Preferably, conjugation to form a covalent bond consists of simply mixing the reactive compounds of the present invention in a suitable solvent in which both the reactive compound and the substance to be conjugated are soluble. The reaction preferably proceeds spontaneously without added reagents at room temperature or below. For those reactive compounds that are photoactivated, conjugation is facilitated by illumination of the reaction mixture to activate the reactive compound. Chemical modification of water-insoluble substances, so that a desired compound-conjugate may be prepared, is preferably performed in an aprotic solvent such as dimethylformamide, dimethylsulfoxide, acetone, ethyl acetate, toluene, or chloroform. Similar modification of water-soluble materials is readily accomplished through the use of the instant reactive compounds to make them more readily soluble in organic solvents.

Preparation of antibody conjugates typically comprises first dissolving the protein to be conjugated in aqueous buffer at about 1-10 mg/mL at room temperature or below. Bicarbonate buffers (pH about 8.3) are especially suitable for reaction with succinimidyl esters, phosphate buffers (pH about 7.2-8) for reaction with thiol-reactive functional groups and carbonate or borate buffers (pH about 9) for reaction with isothiocyanates and dichlorotriazines. The appropriate reactive compound is then dissolved in a nonhydroxylic solvent (usually DMSO or DMF) in an amount sufficient to give a suitable degree of conjugation when added to a solution of the protein to be conjugated. The appropriate amount of compound for any protein or other component is conveniently predetermined by experimentation in which variable amounts of the compound are added to the protein, the conjugate is chromatographically purified to separate unconjugated compound and the compound-protein conjugate is tested in its desired application.

Following addition of the reactive compound to the component solution, the mixture is incubated for a suitable period (typically about 1 hour at room temperature to several hours on ice), the excess compound is removed by gel filtration, dialysis, HPLC, adsorption on an ion exchange or hydrophobic polymer or other suitable means. The compound-conjugate is used in solution or lyophilized. In this way, suitable conjugates can be prepared from antibodies.

Conjugates of polymers, including biopolymers and other higher molecular weight polymers are typically prepared by means well recognized in the art (for example, Brinkley et al., Bioconjugate Chem., 3: 2 (1992)). In these embodiments, a single type of reactive site may be available, as is typical for polysaccharides, or multiple types of reactive sites (e.g. amines, thiols, alcohols, phenols) may be available, as is typical for proteins. Selectivity of labeling is best obtained by selection of an appropriate reactive dye. For example, modification of thiols with a thiol-selective reagent such as a haloacetamide or maleimide, or modification of amines with an amine-reactive reagent such as an activated ester, acyl azide, isothiocyanate or 3,5-dichloro-2,4,6-triazine. Partial selectivity can also be obtained by careful control of the reaction conditions. When modifying polymers with the compounds, an excess of compound is typically used, relative to the expected degree of compound substitution. Any residual, unreacted compound or a compound hydrolysis product is typically removed by dialysis, chromatography or precipitation. Presence of residual, unconjugated dye can be detected by thin layer chromatography using a solvent that elutes the dye away from its conjugate. In all cases it is usually preferred that the reagents be kept as concentrated as practical so as to obtain adequate rates of conjugation.

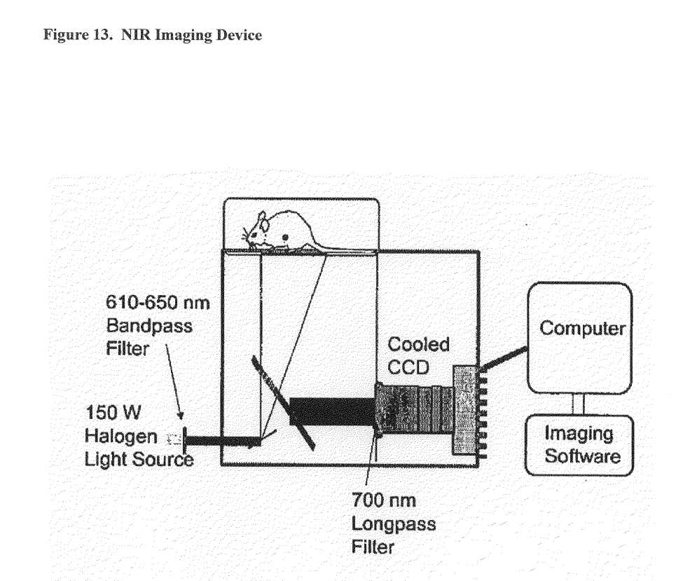

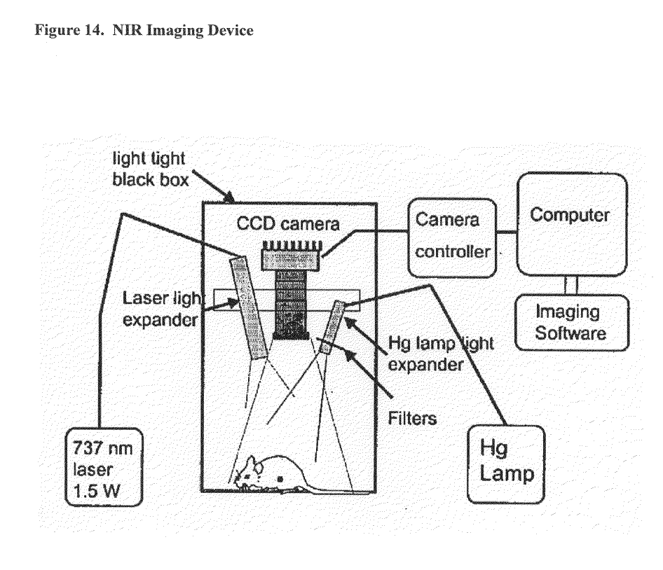

Means for detection of NIR dye labeled conjugates and means for image analysis to localize and quantify the signal produced by such a conjugate at the tumor site are well known in the art. Such means may be separate instruments or incorporated into a single integrated instrument package. Commercial examples of integrated instrument packages include the CRi Maestro™ Imaging Systems (e.g. Maestro™ 2, Maestro™ EX, Maestro DyCE™; Cri, Woburn, Mass., USA); IVIS® Spectrum (Caliper Life Sciences, Hopkinton, Mass., USA). Additionally, examples of instruments that may be used and how to use them are described in the following U.S. Pat. Nos. and Published Applications: 6,615,063; 2008/0218732; 2008/0177140; and 2004/0022731, and also illustrated in FIGS. 13 and 14.

Although the instant invention involves novel methods, general principles of NIR fluorescence, optical image acquisition, and image processing and analysis can be applied in the practice of the invention. For a review of optical imaging techniques, see, e.g., Alfano et al., Ann. NY Acad. Sci., 820:248-270, 1997.

An NIR imaging system useful in the practice of this invention typically includes three basic components: (1) a source of near-infrared or other light of a wavelength suitable to cause the fluorophore of the conjugate to fluoresce, (2) an apparatus for separating or distinguishing emissions from light used for fluorophore excitation, and (3) a detection system. See, e.g., Weissleder et al., Nature Biotechnol., 17:375-8, 1999. For example, an imaging system such as is shown in FIG. 13 can be assembled using a Kodak ImageStation 440 imaging station and an external low-power excitation source. A surface reflectance fluorescent (SRF) imaging device such as this could be used to generate in vivo data for the methods of the invention described herein. The device includes a white light halogen source producing a low intensity light (1 μW/cm² at 650 nm). In surface reflectance imaging, light at a wavelength needed to excite the fluorochrome is applied to the surface of the animal positioned on the glass platen, and an image is made of the Stokes-shifted (“fluorescent”) light. Bandpass filters can be used for wavelength discrimination. More sophisticated systems, such as the one shown in FIG. 14, which features high power laser excitation from the laser (1 mW/cm.sup.2 at 737 nm) and a mercury lamp for light at other wavelengths, more varied filters and an improved CCD camera, can also be used.

Typically, the light source provides monochromatic (or substantially monochromatic) near-infrared light when using NIR fluorophores. The light source can be a suitably filtered white light, e.g., bandpass-filtered light from a broadband source. For example, light from a 150-watt halogen lamp can be passed through a suitable bandpass filter commercially available from Omega Optical (Brattleboro, Vt.). In some embodiments, the light source is a laser. See, e.g., Boas et al., Proc. Natl. Acad. Sci. USA, 91:4887-4891, 1994; Ntziachristos et al., Proc. Natl. Acad. Sci. USA, 97:2767-2772, 2000; Alexander, J. Clin. Laser Med. Surg., 9:416-418, 1991. Information on near-infrared lasers for imaging can also be found on the Internet (e.g., at www.imds.com; Imaging Diagnostic Systems Inc., Fort Lauderdale, Fla., USA) and various other known sources.

A high pass or bandpass filter (700 nm) can be used to separate optical emissions from excitation light. A suitable high pass or bandpass filter is commercially available from Omega Optical (Brattleboro, Vt.). Where the fluorochrome consists of one or more quantum dots, a single excitation wavelength can be used to excite multiple different fluorochromes on a single probe or multiple probes (with different activation sites), and spectral separation with a series of bandpass filters, diffraction grating, or other means can be used to independently read the different activations.

In general, the light detection system can include light-gathering/image-forming and light-detection/image-recording components. Although the light-detection system can be a single integrated device that incorporates both components, the light-gathering/image-forming and light-detection/image-recording components will be discussed separately. However, a recording device may simply record a single (time varying) scalar intensity instead of an image. For example, a catheter-based recording device can record information from multiple sites simultaneously (i.e., an image), or can report a scalar signal intensity that is correlated with location by other means (such as a radio-opaque marker at the catheter tip, viewed by fluoroscopy).

Tomographic approaches to NIR fluorescence and other imaging can also be used. In general, tomographic methods make use of laser light pulses directed through an animal placed in a homogeneously scattering environment; scattered and fluorescent light that has passed through the animal is recorded at numerous positions. Sophisticated modeling algorithms are then applied to localize the source of excited light in the medium. This approach, termed FMT (fluorescence mediated tomography) is described in Ntziachristos et al., Molecular Imaging, 1(2):82-88, 2002 and Ntziachristos et al., Nature Medicine, 8:757-760, 2002.

A particularly useful light-gathering/image-forming component is an endoscope. Endoscopic devices and techniques that have been used for in vivo optical imaging of numerous tissues and organs, including peritoneum (Gahlen et al., J. Photochem. Photobiol., B 52:131-135, 1999), ovarian cancer (Major et al., Gynecol. Oncol., 66:122-132, 1997), colon (Mycek et al., Gastrointest. Endosc., 48:390-394, 1998; Stepp et al., Endoscopy, 30:379-386, 1998), bile ducts (Izuishi et al., Hepatogastroenterology, 46:804-807, 1999), stomach (Abe et al., Endoscopy 32:281-286, 2000), bladder (Kriegmair et al., Urol. Int., 63:27-31, 1999; Riedl et al., J. Endourol., 13:755-759, 1999), and brain (Ward, J. Laser Appl., 10:224-228, 1998) can be employed in the practice of the present invention. Fluorescence endoscopes are also known in the art (Bhunchet et al., Gastrointest. Endosc., 55, 562-571, 2002; Kobayashi et al., Cancer Lett., 165, 155-159, 2001). One of skill in the art would be able to recognize and make any modifications that may be required, e.g., to optimize the emission and detection spectra of the device for use in imaging a particular organ or tissue region.

Other types of light gathering components useful in the invention are catheter-based devices, including fiber optics devices. Such devices are particularly suitable for intravascular imaging. See, e.g., Tearney et al., Science, 276:2037-2039, 1997; Boppart et al., Proc. Natl. Acad. Sci. USA, 94:4256-4261, 1997.

Still other imaging technologies, including phased array technology (Boas et al., Proc. Natl. Acad. Sci. USA, 91:4887-4891, 1994; Chance, Ann. NY Acad. Sci., 838:29-45, 1998), diffuse optical tomography (Cheng et al., Optics Express, 3:118-123, 1998; Siegel et al., Optics Express, 4:287-298, 1999), intravital microscopy (Dellian et al., Br. J. Cancer, 82:1513-1518, 2000; Monsky et al, Cancer Res., 59:4129-4135, 1999; Fukumura et al., Cell, 94:715-725, 1998), and confocal imaging (Korlach et al., Proc. Natl. Acad. Sci. USA, 96:8461-8466, 1999; Rajadhyaksha et al., J. Invest. Dermatol., 104:946-952, 1995; Gonzalez et al., J. Med., 30:337-356, 1999) can be employed in the practice of the present methods.

Any suitable light-detection/image-recording component, e.g., charge-coupled device (CCD) systems or photographic film, can be used in the invention. The choice of light-detection/image-recording component will depend on factors including type of light gathering/image forming component being used. Selecting suitable components, assembling them into a near infrared imaging system, and operating the system is within the ability of a person of ordinary skill in the art.

Any radionuclide known to one of skill in the art which emits or may be caused to emit detectable radiation and that is compatible with in vivo imaging can be used as a radionuclide reporter molecule for the above described EMT-status-detecting conjugates. Highly specific and sensitive labels are provided by radionuclides, which can then be detected, using positron emission tomography (PET) or Single Photon Emission Computed Tomography (SPECT) imaging. The radionuclide reporter of the invention may contain a radionuclide selected from the group consisting of ¹³¹I, ¹²⁵I, ¹²³I, ^{99m}Tc, ¹⁸F, ⁶⁸Ga, ⁶⁷Ga, ⁷²As, ⁸⁹Zr, ⁶⁴Cu, ⁶²Cu, ¹¹¹In, ²⁰³Pb, ¹⁹⁸Hg, ¹¹C, ⁹⁷Ru, ²⁰¹Tl, ⁹⁰Y, ⁴⁷Sc, ⁵¹Cr, ^{177m}Sn, ¹⁶⁷Tm, ¹⁸⁸Re, ¹⁷⁷Lu, ¹⁹⁹Au, ²⁰³Pb and ¹⁴¹Ce.

The energies of the gamma-ray photons emitted by the radioisotopes are unique to each isotope. At the time of their creation, these gamma rays are termed “full energy” or “primary” gamma rays. For the emitted photon to have enough energy to exit the patent's body in sufficient quantities to be able to form an image in a gamma camera, its energy must be above about 60 keV. Gamma rays with energies from 60 to 500 keV usually travel relatively long distances before absorption in soft tissue (several hundred millimeters). For radiotracers in common use, the gamma-ray energies may be as high as about 511 keV. As an example, when Technetium 99 m, an isotope often used in nuclear medicine, decays, 89% of the time a full-energy 140-keV gamma ray is emitted. Natural abundance (“abundance”) or yield refers to the percentage of time that a decay or disintegration of the radioisotope nucleus results in production of the photon of interest, in this case the 140-keV full-energy gamma-ray photon. Indium 111, another commonly used radioisotope, emits 172-keV full-energy gamma rays, with an abundance of 89.6%, and 247-keV full-energy gamma rays, with an abundance of 93.9%.

Gamma-ray emitting radioisotopes also emit characteristic x rays. The emitted x ray is described as “characteristic” because its energy is characteristic of the specific element involved. Characteristic x-ray emissions from radioisotopes used in nuclear medicine are typically of low energies i.e., from about 15 to 30 keV. For example, the radioactive decay of Technetium-99m results in Technetium characteristic x rays of about 19 keV, with an abundance of 7.5%, in addition to the 140 keV gamma ray indicated above. The radioactive decay of Indium 111 results in Cadmium characteristic x-rays of approximately 24 keV, with an abundance of 83.5%. Characteristic x rays of about 20 to 30 keV usually travel only about 30 millimeters or less before absorption in soft tissue. Consequently, these x rays cannot create images in gamma cameras because they are virtually all absorbed by fat, muscle, and skin, but can be detected by the NEOPROBE® device, made by Neoprobe Corporation of Columbus, Ohio, which for example detects both the 27-keV x rays and 35-keV gamma rays from Iodine 125.

The label used is selected according to the means for detection and imaging analysis to be used. For example, radioactive labels, such as Indium-111 (¹¹¹In), Technetium-99m (^{99m}Tc), or Iodine 131 (¹³¹I), can be used for planar scans or for single photon emission computed tomography (SPECT). Also, positron-emitting labels such as Fluorine-18 can be used in positron emission tomography (PET). Additional suitable positron emitting radionuclides for this purpose include ¹¹C, ¹⁵O, ¹³N, ⁷⁵Br, ¹²²I, ¹²⁴I, ⁸²Rb, ⁶⁸ Ga, and ⁶²Cu. Such labels may be incorporated into the conjugate by covalent bonding directly to an atom of the targeting molecule, or the label may be non-covalently or covalently associated with the targeting molecule through a chelating structure or through an auxiliary molecule such as mannitol, gluconate, glucoheptonate, tartrate, and the like. When a chelating structure is used to provide spatial proximity between the label and the targeting molecule, the chelating structure may be directly associated with the targeting molecule or it may be associated with the targeting molecule through an auxiliary molecule such as mannitol, gluconate, glucoheptonate, tartrate, and the like.

Any suitable chelating structure may be used to provide spatial proximity between the radionuclide and the targeting molecule of the agent through covalent or noncovalent association. Many such chelating structures are known in the art. Preferably, the chelating structure is an N₂S₂ structure, an NS₃, structure, an N₄ structure, an isonitrile-containing structure, a hydrazine containing structure, a HYNIC (hydrazinonicotinic acid) group-containing structure, a 2-methylthiolnicotinic acid group-containing structure, a carboxylate group containing structure, and the like. In some cases, chelation can be achieved without including a separate chelating structure, because the radionuclide chelates directly to atom(s) in the targeting moiety, for example to oxygen atoms in various moieties.

The chelating structure, auxiliary molecule, or radionuclide may be placed in spatial proximity to any position of the targeting molecule which does not interfere with the interaction of the targeting molecule with its target site in cardiovascular tissue. Accordingly, the chelating structure, auxiliary molecule, or radionuclide may be covalently or non-covalently associated with any moiety of the targeting molecule except the receptor-binding moiety.

Radionuclides may be placed in spatial proximity to the targeting molecule using known procedures which effect or optimize chelation, association, or attachment of the specific radionuclide to ligands. For example, when ¹²³I, is the radionuclide, the imaging agent may be labeled in accordance with the known radioiodination procedures such as direct radioiodination with chloramine T, radioiodination exchange for a halogen or an organometallic group, and the like. When the radionuclide is ^{99m}Tc, the imaging agent may be labeled using any method suitable for attaching ^{99m}Tc to a ligand molecule. Preferably, when the radionuclide is ^{99m}Tc, an auxiliary molecule such as mannitol, gluconate, glucoheptonate, or tartrate is included in the labeling reaction mixture, with or without a chelating structure. More preferably, ^{99m}Tc is placed in spatial proximity to the targeting molecule by reducing ^{99m}Tc with tin in the presence of mannitol and the targeting molecule. Other reducing agents, including tin tartrate or non-tin reductants such as sodium dithionite, may also be used to make the cardiovascular imaging agent of the invention.

In general, labeling methodologies vary with the choice of radionuclide, the moiety to be labeled and the clinical condition under investigation. Labeling methods using ^{99m}Tc and ¹¹¹In are described for example in Peters, A. M. et al., Lancet 2 946-949 (1986); Srivastava, S. C. et al., Semin. Nrcl. Med. 14(2):68-82 (1984), Sinn, H. et al., Nucl. Med. (Stuttgart) 13:180, 1984), McAfee, J. G. et al., J. Nucl. Med. 17:480-487, 1976, McAfee, J. G. et al., J. Nucl. Med. 17:480-487, 1976; Welch, M. J. et al., J. Nucl. Med. 18:558-562, 1977, McAfee, J. G., et al., Semin. Nucl. Med. 14(2):83, 1984; Thakur, M. L., et al., Semin. Nucl. Med. 14(2):107, 1984; Danpure, H. J. et al., Br. J. Radiol., 54:597-601, 1981; Danpure, H. J. et al., Br. J. Radiol. 55:247-249, 1982; Peters, A. M. et al., J. Nucl. Med. 24:39-44, 1982, Gunter, K. P. et al., Radiology 149:563-566, 1983, and Thakur, M. L. et al., J. Nucl. Med. 26:518-523, 1985.

After the labeling reaction is complete, the reaction mixture may optionally be purified using one or more chromatography steps such as Sep Pack or high performance liquid chromatography (HPLC). Any suitable HPLC system may be used if a purification step is performed, and the yield of reporter conjugate obtained from the HPLC step may be optimized by varying the parameters of the HPLC system, as is known in the art. Any HPLC parameter may be varied to optimize the yield of reporter conjugate of the invention. For example, the pH may be varied, e.g., raised to decrease the elution time of the peak corresponding to the reporter conjugate of the invention.

The radionuclide labeled conjugate formulation is administered in an amount which gives a reliable image, taking into account the nature of the tumor(s) being investigated, size of patient, and such other factors as would be apparent to a person skilled in the art. Where the reporter moiety comprises a metal, generally doses of from 0.001 to 5.0 mmoles of chelated imaging metal ion per kilogram of patient bodyweight are effective to achieve a reliable image. For PET, a suitable amount of a tracer compound is 0.1 to 100 mCi, preferably 1 to 20 mCi.

Means for detection of radioactively labeled conjugates and means for image analysis to localize and quantify the signal produced by such a conjugage at the tumor site are well known in the art. Such means may be separate instruments or incorporated into a single integrated instrument package. Commercial examples include gamma detection devices or probes made by Neoprobe Corporation (Columbus, Ohio); SPECT scanners manufactured by for example Mediso (Budapest, Hungary), GE HealthCare, Philips, GammaMedica (Northridge, Calif.), BioScan Inc. (Washington, D.C.), and Siemens; and PET scanners manufactured by for example Positron Corporation (Fishers, Ind.), Phillips, Cti Molecular Imaging (Knoxville, Tenn.), Crystal Clear Collaboration (http://crystalclear.web.cern.ch/crystalclear), and Siemens. Examples of instruments that may be used and how to use them are also described in the following U.S. Pat. Nos. 4,782,840, 4,801,803, 4,893,013, 5,694,933, and in for example the following articles: Ter-Pogossian, M. M. et al. (1975). “A positron-emission transaxial tomograph for nuclear imaging (PET)”. Radiology 114 (1): 89-98; Phelps, M. E. et al. (1975). “Application of annihilation coincidence detection to transaxial reconstruction tomography”. Journal of Nuclear Medicine 16 (3): 210-224; and Sweet, W. H. and G. L. Brownell (1953). “Localization of brain tumors with positron emitters”. Nucleonics 11: 40-45. Commercially available small animal PET imaging systems are described for example in Larobina, M. et al. Current Medical Imaging Reviews, 2006, 2, 187-192.

In an alternative embodiment of any of the methods or compositions of the invention described herein, the “EMT-status-detecting conjugate” is a probe or complex wherein instead of an antibody capable of binding specifically to an EMT-status biomarker, another “targeted carrier molecule” that can bind specifically to an EMT-status biomarker is used. This “targeted carrier molecule” can be any biological or non-biological molecule that has a specific binding partner (i.e. an EMT-status biomarker) in the tumor of a patient and can travel relatively freely in circulating blood or lymph to reach the tumor. Examples of “targeted carrier molecules” include AFFIBODY® molecules, peptides, aptamers or peptidomimetics that bind specifically to an EMT-status biomarker, such as E-cadherin. Such “targeted carrier molecules” can be labeled with a reporter molecule in a similar way as antibodies, with for example an NIR dye or radionuclide.

One particular preferred alternate “targeted carrier molecule” is the AFFIBODY® molecule (Affibody AB, Sweden) (Nygren P. A., (2008) FEBS J. 275(11):2668-76; Nord, K., et al. (1997) Nature Biotechnol. 15: 772-777; Tolmachev V. et al. (2007) Expert Opin Biol Ther. 2007; 7(4):555-68.; Lendel C., et al. (2006) J Mol. Biol. 2006; 359(5):1293-304). AFFIBODY® molecules are non-immunoglobulin binding proteins developed using combinatorial protein engineering principles, that are functionally selected from libraries of a small (6 kDa), non-cysteine three-helix bundle domain used as a scaffold. Similar to antibodies, they can readily be selected for any desired epitope or antigen, and thus preparation of AFFIBODY® molecules that bind to the extracellular domains of EMT biomarkers is a routine procedure well known to those of skill in the art (for applicable methods, see for example International Patent Applications WO 05/003156, WO 2009/019117 and WO 05/000883, which disclose methods of preparing and using AFFIBODY® molecules, including for example those that bind to the proteins HER2, IGF-1R and insulin). High-affinity AFFIBODY® molecules have been selected towards a large number of targets (e.g. EGFR, Fibrinogen, HER-2, HAS, IgA, IgE, IgM, IL-8, Insulin, TNF-a, Transferrin, Transthyretin) for use in a variety of applications, such as bioseparation, diagnostics, functional inhibition, viral targeting, and in vivo tumor imaging/therapy (Orlova A., et al. (2007) Cancer Res. 67(5):2178-86; Lundberg E., et al. (2007) Journal of Immunological Methods 319:53-63; Orlova A., et al. (2006) Cancer Res. 66(8):4339-48; Tolmachev V., et al. (2007) Cancer Res. 2007 Mar. 15; 67(6):2773-82; Cheng Z., et al. (2008) J Nucl Med. 49(5):804-13; Engfeldt T., et al. (2007) Eur J Nucl Med Molecular Imaging, 34(5):722-33; Engfeldt T. et al. (2007) Eur J Nucl Med Molecular Imaging, 2007, November; 34(11):1843-53.; Kramer-Marek G., et al (2007) Eur J Nucl Med Molecular Imaging, December 22). Due to their small size it is also possible to produce functional AFFIBODY® molecules by chemical synthesis production routes, which is advantageous for the site-specific introduction of various labels and radionuclide chelators. The small size of AFFIBODY® molecules also confers them with favorable pharmacokinetic properties when used in vivo, including both rapid tumor targeting and rapid clearance from the circulation, which is advantageous when used in the methods of the instant invention when short-lived radionuclides are used as reporter molecules (e.g. ⁶⁸Ga or ¹⁸F, for PET imaging).

This invention thus also provides a method of determining in situ the EMT status of the cells of a tumor in a patient, comprising: (a) providing an EMT-status-detecting conjugate comprising an AFFIBODY® molecule that binds to an EMT-status biomarker, and a reporter molecule that produces a detectable signal; (b) introducing said conjugate into a patient with a tumor, such that the conjugate contacts the EMT-status biomarker of the cells of the tumor; (c) employing a means for detection of the signal from the conjugate at the tumor site; and (d) using a means for image analysis to localize and quantify the signal from the conjugate at the tumor site, a positive signal indicating the presence of epithelial tumor cells if the EMT-status biomarker is an epithelial biomarker, and mesenchymal tumor cells if the EMT-status biomarker is a mesenchymal biomarker.

This invention also provides a method of identifying an agent that inhibits the cells of a tumor in a patient from undergoing an epithelial to mesenchymal transition, wherein the patient is an animal model of cancer comprising: (a) introducing a test agent that into said patient such that the agent contacts the cells of the tumor; (b) contacting the patient with an agent capable of inducing the cells of the tumor to undergo EMT; (c) providing an EMT-status-detecting conjugate comprising an AFFIBODY® molecule that binds to an EMT-status biomarker, and a reporter molecule that produces a detectable signal; (d) introducing said conjugate into the patient, such that the conjugate contacts the EMT-status biomarker of the cells of the tumor; (e) employing a means for detection of the signal from the conjugate at the tumor site; (f) using a means for image analysis to localize and quantify the signal from the conjugate at the tumor site, a positive signal indicating the presence of epithelial tumor cells if the EMT-status biomarker is an epithelial biomarker, and mesenchymal tumor cells if the EMT-status biomarker is a mesenchymal biomarker, and (g) comparing the signal in (f) with that produced in a similarly treated patient but that was not treated with the test agent as in (a), thus identifying whether said test agent is an agent that can inhibit tumor cells from undergoing an epithelial to mesenchymal transition.

This invention also provides a method of identifying an agent that inhibits the cells of a tumor in a patient from undergoing an epithelial to mesenchymal transition, comprising: (a) introducing a test agent that into said patient such that the agent contacts the cells of the tumor; (b) providing an EMT-status-detecting conjugate comprising an AFFIBODY® molecule that binds to an EMT-status biomarker, and a reporter molecule that produces a detectable signal; (c) introducing said conjugate into the patient, such that the conjugate contacts the EMT-status biomarker of the cells of the tumor; (d) employing a means for detection of the signal from the conjugate at the tumor site; (e) using a means for image analysis to localize and quantify the signal from the conjugate at the tumor site, a positive signal indicating the presence of epithelial tumor cells if the EMT-status biomarker is an epithelial biomarker, and mesenchymal tumor cells if the EMT-status biomarker is a mesenchymal biomarker, and (f) comparing the signal in (e) with that produced in a similarly treated patient but that was not treated with the test agent as in (a), thus identifying whether said test agent is an agent that can inhibit tumor cells from undergoing an epithelial to mesenchymal transition.

This invention also provides a composition comprising an EMT-status-detecting conjugate comprising an AFFIBODY® molecule that binds to an extracellular domain of an EMT-status biomarker and a reporter molecule that produces a detectable signal, for use in a method of determining in situ the EMT status of the cells of a tumor in a patient. In a preferred embodiment the AFFIBODY® molecule and the reporter molecule are covalently bound. The EMT-status biomarker may be an epithelial or a mesenchymal biomarker. Examples of suitable EMT-status biomarkers are listed herein. In one embodiment the EMT-status biomarker is E-cadherin. The reporter molecule may be for example an NIR dye or a radionuclide, of which several suitable examples are listed herein. In one embodiment the reporter molecule comprises Alexa Fluor 680 fluorescent dye.

The EMS-status-detecting conjugate can be introduced into the patient by any means known for uptake into the body, by either enteral or parenteral administration, which includes, but is not limited to, orally or intravenously. In one aspect the EMS-status-detecting conjugate is introduced into the patient intravenously by injection with a needle into a vein (such as the tail vein, if the patient is a mouse or rat). Once in circulation, the conjugate travels relatively freely until encountering the target EMT-status antigen, wherein the conjugate associates non-covalently with the target and is sequestered at the tumor until being cleared by normal bodily processes. When the patient is a mouse xenograft model, amounts of conjugate administered are from between about 5 μg to about 100 μg. For larger animals corresponding larger amounts are used relative to the weight of the animal.

Another embodiment further comprises a step of: incubating the contacted patient for a period of time sufficient for the conjugate to contact the target EMT-status antigen. More particularly, the period of time is at least 30 min, 90 min, 2 hr, 6 hr, 24 hr, 48 hr, 3 days, 4 days, 7 days, 9 days, or longer.

In an additional embodiment the step of using a means for image analysis to localize and quantify the signal comprises a step of: transmitting data onto a computer processor, wherein the data represents the detectable signal from the EMS-status-detecting conjugate; and performing an analysis of the data with the computer processor to determine a result indicating the presence, amount or location of the target EMT-status antigen.

Another embodiment further comprises a display unit or printout which visually displays the result. Another embodiment provides a method for identifying the EMT-status of a tumor in a patient, the method comprising: a) providing a dye conjugate comprising a NIR dye and an antibody that binds to a target EMT-status antigen; b) introducing the dye conjugate into the patient to form a contacted patient; c) illuminating the contacted patient with an appropriate wavelength to form an illuminated patient; and d) observing the illuminated patient wherein the tumor EMT-status is identified; wherein the target EMT-status antigen is an EMT-status biomarker having an extracellular domain.

When the EMS-status-detecting conjugate contains an NIR reporter, and requires illumination to produce a detectable signal, the patient may be illuminated at any time after the conjugate has been introduced into the body. In one aspect the body is illuminated min, 30 min, 90 min, 2 hr, 6 hr, 24 hr, 48 hr, 3 days, 4 days, 7 days, 9 days or longer post-injection. The instrument used for illumination and visualization can be any instrument known in the art for non-in vivo or in vivo imaging.

Due to the advantageous properties and the simplicity of use of the instant EMT-status-detecting conjugates, they are particularly useful in the formulation of a kit for non-invasive in vivo imaging. In one embodiment the kits comprise instant EMT-status-detecting conjugate and instructions for in vivo imaging. In another embodiment the kits comprise a reactive NIR reporter molecule, dye or particle, an anti-EMT-status biomarker antibody, instructions for conjugating the reactive reporter molecule to the anti-EMT-status biomarker antibody and instructions for in vivo imaging. In yet another embodiment, the kits comprise a reactive reporter molecule, instructions for conjugating the reactive reporter molecule to an anti-EMT-status biomarker antibody and instructions for in vivo imaging. One particular embodiment provides a kit for imaging a target EMT-status antigen in a patient comprising: a) a dye conjugate comprising a NIR dye and an antibody that binds to a target EMT-status antigen; and b) instructions for imaging the target EMT-status antigen. More particularly, the kit further comprises at least one of: a needle, imaging software, reagents, buffers, diluents, excipients, additional dyes or antibodies. In a preferred embodiment of the kit, the target EMT-status antigen is E-cadherin.

In any of the methods of this invention where induction of EMT by exogenous agents is required, the tumor cells (e.g. tumor cells in a xenograft animal model) may be induced to undergo EMT using any agent known to induce EMT for the given tumor cell type. For example, the protein TGF-beta may be employed. “TGFbeta”, as used herein means TGFbeta-1, TGFbeta-2, or TGFbeta-3 (Schmierer, B. and Hill, C. (2007) Nat Rev Mol Cell Biol. 8(12):970-82), or heterodimers thereof. The TGF-beta is preferably human, but TGF-beta from other species that are active in promoting EMT in the tumor cells may also be used (e.g. from mouse, rat, pig, rabbit, chicken, bovine). Additional EMT-inducing agents that may be used include for example, the proteins HGF (Lamorte, L. et al (2002) Mol Biol Cell. 13(5): p. 1449-61), Hedgehog (Feldmann, G. et al (2007) Cancer Res. 67(5): p. 2187-96), Wnt (Yook, J. et al (2006) Nat Cell Biol. 8(12): p. 1398-406), IL-1 (Chaudhuri, V. et al (2007) J Cutan Pathol. 34(2): p. 146-53), Oncostatin M (Pollack, V. et al (2007) Am J Physiol Renal Physiol. 293(5): p. F1714-26), EGF (Solic, N. and Davies, D. (1997) Exp Cell Res. 234(2): p. 465-76), Amphiregulin (Chung, (2005) E. Exp Cell Res. 309(1): p. 149-60), HB-EGF (Wang, F. et al (2007) Cancer Res. 67(18): p. 8486-93), MSP (Camp, E., (2007) Cancer. 109(6): p. 1030-9), Wnt5a (Dissanayake, S. (2007) J Biol. Chem. 282(23): p. 17259-71; Ripka, S. (2007) Carcinogenesis. 28(6): p. 1178-87), and TNF-alpha (Bates, R. and Mercurio, A. (2003) Mol Biol Cell. 14(5): p. 1790-800). Alternatively, the tumor cells may be engineered to indicibly-express a protein that causes the cells to undergo EMT, e.g. Snail, Zeb1 or TGF-beta. The inducible expression may be for example, a tet-on or tet-off system in which the level of Snail, and thus EMT induction, can be modulated by the presence or absence of a tetracycline analogue such as doxycycline (e.g. see Guaita, S. et al (2002) J. Biol. Chem. 277(42):39209-39216). Examples of tumor cells that may be used in animal models, and may be engineered to indicibly-express a protein that causes the cells to undergo EMT, include the NSCLC cell line H358, breast cancer cell lines MCF7 (Hiscox, X. (2006) Int J. Cancer. 118(2): p. 290-301), T47D (Jorcyk, C. et al (2006) Cytokine. 33(6): p. 323-36), and MDA-MB-468 (Lester, R. (2007) J. Cell Biol. 178(3): p. 425-36), pancreatic cancer cell lines L3.6 μl (Yang, A. et al (2006) Cancer Res. 66(1): p. 46-51), PANC-1, COLO-357, and IMIM-PC1 (Ellenrieder, V. (2001) Cancer Res. 61(10): p. 4222-8), and colon cancer cell lines HT29 (Yang, L. (2006) Cell. 127(1): p. 139-55), LIM 1863 (Bates, R. et al (2004) Exp Cell Res. 299(2):315-24), and KM12L4 (Yang, A. (2006) Clin Cancer Res. 12(14 Pt 1):4147-53). For example, see U.S. patent application 61/068,612 for a description of how to make and use an inducible H358 tumor cell model of EMT.

Many protein biomarkers are known whose level of expression or activity is indicative of the EMT status of tumor cells (i.e. EMT-status biomarkers) (e.g. see US Patent Application Publication 2007/0212738; U.S. Patent Application 60/923,463; U.S. Patent Application 60/997,514). Such biomarker proteins tend to be classified as epithelial or mesenchymal, due to their characteristic association with the particular stage of EMT. In any of the methods or compositions described herein the EMT-status biomarker whose expression level is indicative of the EMT status of the sample tumor cells may be any epithelial cell biomarker that has an extracellular domain. Epithelial cell biomarkers include for example E-cadherin (e.g. human E-cadherin, CDH-1, NCBI GeneID number 999), cytokeratin 8, cytokeratin 18, P-cadherin, erbB3, Brk, γ-catenin, α1-catenin, α2-catenin, α3-catenin, connexin 31, plakophilin 3, stratifin 1, laminin alpha-5, and ST14. Examples of additional epithelial markers that can be used in any of the methods of this invention include phospho-14-3-3 epsilon, 14-3-3 gamma (KCIP-1), 14-3-3 sigma (Stratifin), 14-3-3 zeta/delta, phosphoserine/threonine phosphatase 2A, 4F2hc(CD98 antigen), adenine nucleotide translocator 2, annexin A3, ATP synthase beta chain, phospho-insulin receptor substrate p53/p54, Basigin (CD147 antigen), phospho-CRK-associated substrate (p130Cas), Bcl-X, phospho-P-cadherin, phospho-calmodulin (CaM), Calpain-2 catalytic subunit, Cathepsin D, Cofilin-1, Calpain small subunit 1, Catenin beta-1, Catenin delta-1 (p120 catenin), Cystatin B, phospho-DAZ-associated protein 1, Carbonyl reductase [NADPH], Diaphanous-related formin 1 (DRF1), Desmoglein-2, Elongation factor 1-delta, phospho-p185erbB2, Ezrin (p81), phospho-focal adhesion kinase 1, phospho-p94-FER (c-FER)., Filamin B, phospho-GRB2-associated binding protein 1, Rho-GDI alpha, phospho-GRB2, GRP 78, Glutathione S-transferase P, 3-hydroxyacyl-CoA dehydrogenase, HSP 90-alpha, HSP70.1, eIF3 p110, eIF-4E, Leukocyte elastase inhibitor, Importin-4, Integrin alpha-6, Integrin beta-4, phospho-Cytokeratin 17, Cytokeratin 19, Cytokeratin 7, Casein kinase I, alpha, Protein kinase C, delta, Pyruvate kinase, isozymes M1/M2, phospho-Erbin, LIM and SH3 domain protein 1 (LASP-1), 4F21c (CD98 light chain), L-lactate dehydrogenase A chain, Galectin-3, Galectin-3 binding protein, phospho-LIN-7 homolog C, MAP (APC-binding protein EB1), Maspin precursor (Protease inhibitor 5), phospho-Met tyrosine kinase (HGF receptor), Mixed-lineage leukemia protein 2, Monocarboxylate transporter 4, phospho-C-Myc binding protein (AMY-1), Myosin-9, Myosin light polypeptide 6, Nicotinamide phosphoribosyltransferase, Niban-like protein (Meg-3), Ornithine aminotransferase, phospho-Occludin, Ubiquitin thiolesterase, PAF acetylhydrolase IB beta subunit, phospho-partitioning-defective 3 (PAR-3), phospho-programmed cell death 6-interacting protein, phospho-Programmed cell death protein 6, Protein disulfide-isomerase, phospho-plakophilin-2, phospho-plakophilin-3, Protein phosphatase 1, Peroxiredoxin 5, Proteasome activator complex subunit 1, Prothymosin alpha, Retinoic acid-induced protein 3, phospho-DNA repair protein REV 1, Ribonuclease inhibitor, RuvB-like 1, S-100P, S-100L, Calcyclin, S100C, phospho-Sec23A, phospho-Sec23B, Lysosome membrane protein II (LIMP II), p60-Src, phospho-Amplaxin (EMS1), SLP-2, Gamma-synuclein, Tumor calcium signal transducer 1, Tumor calcium signal transducer 2, Transgelin-2, Transaldolase, Tubulin beta-2 chain, Translationally controlled (TCTP), Tissue transglutaminase, Transmembrane protein Tmp21, Ubiquitin-conjugating enzyme E2 N, UDP-glucosyltransferase 1, phospho-p61-Yes, phospho-Tight junction protein ZO-1, AHNAK (Desmoyokin), phospho-ATP synthase beta chain, phospho-ATP synthase delta, Cold shock domain protein E1, Desmoplakin III, Plectin 1, phospho-Nectin 2 (CD112 antigen), phospho-p185-Ron, phospho-SHC1, E-cadherin, Brk, γ-catenin, α1-catenin, α2-catenin, α3-catenin, keratin 8, keratin 18, connexin 31, plakophilin 3, stratafin 1, laminin alpha-5 and ST14, and other epithelial biomarkers known in the art (see for example, US Patent Application Publication 2007/0212738; U.S. Patent Application 60/923,463; U.S. Patent Application 60/997,514). Additionally, any other epithelial cell biomarkers known in that art (e.g. see US Patent Application Publication 2007/0212738; U.S. Patent Application 60/923,463; U.S. Patent Application 60/997,514), described herein, or yet to be described, may be used in the methods of the invention described herein if it has an extracellular domain.

In any of the methods or compositions described herein the EMT-status biomarker whose expression level is indicative of the EMT status of the tumor cells may also be a mesenchymal cell biomarker that has an extracellular domain. Mesenchymal biomarkers include for example: MHC class I antigen A*1, Acyl-CoA desaturase, LANP-like protein (LANP-L), Annexin A6, ATP synthase gamma chain, BAG-family molecular chaperone regulator-2, phospho-Bullous pemphigoid antigen, phospho-Protein Clorf77, CDK1 (cdc2), phospho-Clathrin heavy chain 1, Condensin complex subunit 1,3,2-trans-enoyl-CoA isomerase, DEAH-box protein 9, phospho-Enhancer of rudimentary homolog, phospho-Fibrillarin, GAPDH muscle, GAPDH liver, Synaptic glycoprotein SC2, phospho-Histone H1.0, phospho-Histone H1.2, phospho-Histone H1.3, phospho-Histone H1.4, phospho-Histone H1.5, phospho-Histone H1x, phospho-H1stone H2AFX, phospho-H1stone H2A.o, phospho-H1stone H2A.q, phospho-H1stone H2A.z, phospho-Histone H2B.j, phospho-Histone H2B.r, phospho-Histone H4, phospho-HMG-17-like 3, phospho-HMG-14, phospho-HMG-17, phospho-HMGI-C, phospho-HMG-I/HMG-Y, phospho-Thyroid receptor interacting protein 7 (TRIP7), phospho-hnRNP H3, hnRNP C1/C2, hnRNP F, phospho-hnRNP G, eIF-5A, NFAT 45 kDa, Importin beta-3, cAMP-dependent PK1a, Lamin B1, Lamin A/C, phospho-Laminin alpha-3 chain, L-lactate dehydrogenase B chain, Galectin-1, phospho-Fez1, Hyaluronan-binding protein 1, phospho-Microtubule-actin crosslinking factor 1, Melanoma-associated antigen 4, Matrin-3, Phosphate carrier protein, Myosin-10, phospho-N-acylneuraminate cytidylyltransferase, phospho-NHP2-like protein 1, H/ACA ribonucleoprotein subunit 1, Nucleolar phosphoprotein p130, phospho-RNA-binding protein Nova-2, Nucleophosmin (NPM), NADH-ubiquinone oxidoreductase 39 kDa subunit, phospho-Polyadenylate-binding protein 2, Prohibitin, Prohibitin-2, Splicing factor Prp8, Polypyrimidine tract-binding protein 1, Parathymosin, Rab-2A, phospho-RNA-binding protein Raly, Putative RNA-binding protein 3, phospho-60S ribosomal protein L23, hnRNP A0, hnRNP A2/B1, hnRNP A/B, U2 small nuclear ribonucleoprotein B, phospho-Ryanodine receptor 3, phospho-Splicing factor 3A subunit 2, snRNP core protein D3, Nesprin-1, Tyrosine-tRNA ligase, phospho-Tankyrase 1-BP, Tubulin beta-3, Acetyl-CoA acetyltransferase, phospho-bZIP enhancing factor BEF (Aly/REF; Tho4), Ubiquitin, Ubiquitin carboxyl-terminal hydrolase 5, Ubiquinol-cytochrome c reductase, Vacuolar protein sorting 16, phospho-Zinc finger protein 64, phospho-AHNAK (Desmoyokin), ATP synthase beta chain, ATP synthase delta chain, phospho-Cold shock domain protein E1, phospho-Plectin 1, Nectin 2 (CD112 antigen), p185-Ron, SHC1, vimentin, fibronectin, fibrillin-1, fibrillin-2, collagen alpha-2(IV), collagen alpha-2(V), LOXL1, nidogen, C11orf9, tenascin, N-cadherin, embryonal EDB⁺ fibronectin, tubulin alpha-3 and epimorphin.

Many of the biomarkers listed herein above have been identified as being altered in expression level after EMT in US Patent Application Publication 2007/0212738, the contents of which are incorporated herein by reference; US Published Application 2006/0211060 (filed Mar. 16, 2006); Thomson, S. et al. (2005) Cancer Res. 65(20) 9455-9462; and Yauch, R. L. et al. (2005) Clin. Can. Res. 11(24) 8686-8698).

Additional EMT-status biomarkers that may be used in any of the methods or compositions of the instant invention include the epithelial biomarkers ERB-B3 (e.g. human ERBB3 (v-erb-b2 erythroblastic leukemia viral oncogene homolog 3), NCBI GeneID number 2065), S100-P (e.g. human S100P (S100 calcium binding protein P), NCBI GeneID number 6286), S100-A6 (e.g. human S100A6 (S100 calcium binding protein A6), NCBI GeneID number 6277), TACD1 (e.g. human EPCAM (epithelial cell adhesion molecule), NCBI GeneID number 4072), CD98 (e.g. human SLC3A2 (solute carrier family 3 (activators of dibasic and neutral amino acid transport), member 2), NCBI GeneID number 6520), MUC1 (e.g. human MUC1 (mucin 1, cell surface associated), NCBI GeneID number 4582), and ZO-1 (e.g. human TJP1 (tight junction protein 1 (zona occludens 1)), NCBI GeneID number 7082), and the mesenchymal biomarkers EFNB2 (e.g. human EFNB2 (ephrin-B2), NCBI GeneID number 1948), FLRT3 (e.g. human FLRT3 (fibronectin leucine rich transmembrane protein 3), NCBI GeneID number 23767), SPARC (e.g. human SPARC (secreted protein, acidic, cysteine-rich (osteonectin)), NCBI GeneID number 6678), and CD44 (e.g. human CD44 (CD44 molecule (Indian blood group)), NCBI GeneID number 960).

The NCBI GeneID numbers listed herein are unique identifiers of the gene from the NCBI Entrez Gene database record (National Center for Biotechnology Information (NCBI), U.S. National Library of Medicine, 8600 Rockville Pike, Building 38A, Bethesda, Md. 20894; Internet address http://www.ncbi.nlm.nih.gov/). They are used herein to unambiguously identify gene products that are referred to elsewhere in the application by names and/or acronyms. Proteins expressed by genes thus identified represent proteins that may be used in the methods of this invention, and the sequences of these proteins, including different isoforms, as disclosed in NCBI database (e.g. GENBANK®) records are herein incorporated by reference.

It is a simple matter for the skilled artisan to determine whether an epithelial or mesenchymal biomarker protein, or a portion thereof, is exposed on the cell surface, i.e. has an extracellular domain. For example, immunological methods may be used to detect such proteins on whole cells, or well known computer-based sequence analysis methods may be used to predict the presence of at least one extracellular domain (i.e. including both secreted proteins and proteins having at least one cell-surface domain). Expression of a biomarker protein having at least one portion which is displayed on the surface of a cell which expresses it may be detected in vivo, without biopsy and lysing the tumor cell, using a labeled antibody which binds specifically with a cell-surface domain of the protein.

In any of the methods described herein, multiple biomarker level determinations can also be used to assess EMT status, potentially providing a more reliable assessment. For example, an epithelial and a mesenchymal biomarker level may be assessed, the reciprocal changes in each providing internal confirmation that EMT has occurred. For each biomarker determination, EMT-status-detecting conjugates can be chosen where the detectable signals from the reporter molecules can be evaluated independently and without interference from each other. For example, for NIR dye reporters, two or more reporters can be chosen that have emission maxima that are sufficiently separated that they can be read independently from labeling in the same tumor.

Tumor cells in any of the methods of the instant invention described herein, including when a the patient has cancer or a tumor, whether a human or non-human animal patient with cancer, or an experimental animal implanted with tumor cells, may be tumor cells from any of the following tumors or cancers: NSCL, breast, colon, or pancreatic cancer, lung cancer, small cell lung carcinoma, bronchioloalveolar cell lung cancer, bone cancer, skin cancer, cancer of the head or neck, epithelial carcinoma, cutaneous or intraocular melanoma, uterine cancer, cervical cancer, ovarian cancer, rectal cancer, colorectal cancer, cancer of the anal region, stomach cancer, gastric cancer, uterine cancer, carcinoma of the fallopian tubes, carcinoma of the endometrium, adenocarcinoma, sweat gland carcinoma, sebaceous gland carcinoma, papillary carcinoma, papillary adenocarcinomas, Ewing's tumor, cystadenocarcinoma, medullary carcinoma, bronchogenic carcinoma, choriocarcinoma, seminoma, embryonal carcinoma, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, ovarian cancer, cancer of the penis, prostate cancer, cancer of the bladder, testicular tumor, cancer of the ureter, cancer of the kidney, renal cell carcinoma, carcinoma of the renal pelvis, mesothelioma, hepatocellular cancer, biliary cancer, hepatoma, bile duct carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, leiomyosarcoma, rhabdomyosarcoma, rhabdosarcoma, Wilms' tumor, astrocytoma, Kaposi's sarcoma, medulloblastoma, craniopharyngioma, ependymoma, pinealoma, hemangioblastoma, acoustic neuroma, oligodendroglioma, meningioma, neuroblastoma, retinoblastoma, lymphocytic lymphomas, basal cell carcinoma, neoplasms of the central nervous system (CNS), spinal axis tumors, glioma, brain stem glioma, glioblastoma multiforme, schwannomas, ependymomas, meningiomas, squamous cell carcinomas, pituitary adenomas, including refractory versions of any of the above cancers, or a combination of one or more of the above cancers. When the patient is an experimental animal (e.g. a nude mouse) with a tumor xenograft, the tumor cells may be human tumor cells, or tumor cells from another non-human animal (e.g. dog, cat, bovine, horse, pig, mouse, rat etc.).

The term “refractory” as used herein is used to define a cancer for which treatment (e.g. chemotherapy drugs, biological agents, and/or radiation therapy) has proven to be ineffective. A refractory cancer tumor may shrink, but not to the point where the treatment is determined to be effective. Typically however, the tumor stays the same size as it was before treatment (stable disease), or it grows (progressive disease). The treatment could for example be with any of the list of therapeutic agents described herein.

For any methods described herein where an EGFR kinase inhibitor is used, an example of a preferred EGFR kinase inhibitor is erlotinib, including pharmacologically acceptable salts or polymorphs thereof. In these methods one or more additional anti-cancer agents or treatments can be co-administered simultaneously or sequentially with the EGFR kinase inhibitor, as judged to be appropriate by the administering physician given the prediction of the likely responsiveness of the patient to the EGFR kinase inhibitor, or response to initial therapy, in combination with any additional circumstances pertaining to the individual patient.

For any methods described herein where an IGF-1R kinase inhibitor is used, an example of a preferred EGFR kinase inhibitor is OSI-906, including pharmacologically acceptable salts or polymorphs thereof. In these methods one or more additional anti-cancer agents or treatments can be co-administered simultaneously or sequentially with the IGF-1R kinase inhibitor, as judged to be appropriate by the administering physician given the prediction of the likely responsiveness of the patient to the IGF-1R kinase inhibitor, or response to initial therapy, in combination with any additional circumstances pertaining to the individual patient.

The present invention also provides a method for treating tumors or tumor metastases in a patient, comprising the steps of diagnosing a patient's likely responsiveness to an EGFR or IGF-1R kinase inhibitor by using any of the in situ methods of this invention to identify the patient as one who has a tumor which has not undergone an EMT and thus is likely to be relatively sensitive to an EGFR or IGF-1R kinase inhibitor as a single agent, and administering to said patient simultaneously or sequentially a therapeutically effective amount of an EGFR and/or an IGF-1R kinase inhibitor.

It will be appreciated by one of skill in the medical arts that the exact manner of administering to said patient of a therapeutically effective amount of an EGFR and/or IGF-1 R kinase inhibitor following a diagnosis of a patient's likely responsiveness to an EGFR or IGF-1R kinase inhibitor, using the imaging methods described herein to determine EMT status, will be at the discretion of the attending physician. The mode of administration, including dosage, combination with other anti-cancer agents, timing and frequency of administration, and the like, may be affected by the diagnosis of a patient's likely responsiveness to an EGFR or IGF-1R kinase inhibitor, as well as the patient's condition and history. Thus, even patients diagnosed with tumors predicted to be relatively insensitive to an EGFR and/or IGF-1R kinase inhibitor as a single agent may still benefit from treatment with an EGFR and/or IGF-1R kinase inhibitor, optionally in combination with other anti-cancer agents, or other agents that may alter a tumor's sensitivity to EGFR or IGF-1R kinase inhibitors.

The present invention further provides the methods described herein for treating tumors or tumor metastases in a patient comprising administering to the patient simultaneously or sequentially a therapeutically effective amount of an EGFR and/or IGF-1R kinase inhibitor, with the addition of one or more other cytotoxic, chemotherapeutic, or anti-cancer agents, or compounds that enhance the effects of such agents. In the context of this invention, other anticancer agents includes, for example, other cytotoxic, chemotherapeutic or anti-cancer agents, or compounds that enhance the effects of such agents, anti-hormonal agents, angiogenesis inhibitors, tumor cell pro-apoptotic or apoptosis-stimulating agents, signal transduction inhibitors, anti-proliferative agents, anti-HER2 antibody or an immunotherapeutically active fragment thereof, anti-proliferative agents, COX II (cyclooxygenase II) inhibitors, and agents capable of enhancing antitumor immune responses.

In the context of this invention, other cytotoxic, chemotherapeutic or anti-cancer agents, or compounds that enhance the effects of such agents, include, for example: alkylating agents or agents with an alkylating action, such as cyclophosphamide (CTX; e.g. CYTOXAN®), chlorambucil (CHL; e.g. LEUKERAN®), cisplatin (CisP; e.g. PLATINOL®) busulfan (e.g. MYLERAN®), melphalan, carmustine (BCNU), streptozotocin, triethylenemelamine (TEM), mitomycin C, and the like; anti-metabolites, such as methotrexate (MTX), etoposide (VP16; e.g. VEPESID®), 6-mercaptopurine (6 MP), 6-thiocguanine (6TG), cytarabine (Ara-C), 5-fluorouracil (5-FU), capecitabine (e.g. XELODA®), dacarbazine (DTIC), and the like; antibiotics, such as actinomycin D, doxorubicin (DXR; e.g. ADRIAMYCIN®), daunorubicin (daunomycin), bleomycin, mithramycin and the like; alkaloids, such as vinca alkaloids such as vincristine (VCR), vinblastine, and the like; and other antitumor agents, such as paclitaxel (e.g. TAXOL®) and pactitaxel derivatives, the cytostatic agents, glucocorticoids such as dexamethasone (DEX; e.g. DECADRON®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: amifostine (e.g. ETHYOL®), dactinomycin, mechlorethamine (nitrogen mustard), streptozocin, cyclophosphamide, lomustine (CCNU), doxorubicin lipo (e.g. DOXIL®), gemcitabine (e.g. GEMZAR®), daunorubicin lipo (e.g. DAUNOXOME®), procarbazine, mitomycin, docetaxel (e.g. TAXOTERE®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil.

As used herein, the term “anti-hormonal agent” includes natural or synthetic organic or peptidic compounds that act to regulate or inhibit hormone action on tumors. Antihormonal agents include, for example: steroid receptor antagonists, anti-estrogens such as tamoxifen, raloxifene, aromatase inhibiting 4(5)-imidazoles, other aromatase inhibitors, 42-hydroxytamoxifen, trioxifene, keoxifene, LY 117018, onapristone, and toremifene (e.g. FARESTON®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (leuteinizing hormone-releasing hormone); the LHRH agonist goserelin acetate, commercially available as ZOLADEX® (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N-6-(3-pyridinylcarbonyl)-L-lysyl-N-6-(3-pyridinylcarbonyl)-D-lysyl-L-leucyl-N-6-(1-methylethyl)-L-lysyl-L-proline (e.g ANTIDE®, Ares-Serono); the LHRH antagonist ganirelix acetate; the steroidal anti-androgens cyproterone acetate (CPA) and megestrol acetate, commercially available as MEGACE® (Bristol-Myers Oncology); the nonsteroidal anti-androgen flutamide (2-methyl-N-[4,20-nitro-3-(trifluoromethyl) phenylpropanamide), commercially available as EULEXIN® (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR, RXR, TR, VDR, and the like.

Anti-angiogenic agents include, for example: VEGFR inhibitors, such as SU-5416 and SU-6668 (Sugen Inc. of South San Francisco, Calif., USA), or as described in, for example International Application Nos. WO 99/24440, WO 99/62890, WO 95/21613, WO 99/61422, WO 98/50356, WO 99/10349, WO 97/32856, WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); angiozyme, a synthetic ribozyme from Ribozyme (Boulder, Colo.) and Chiron (Emeryville, Calif.); and antibodies to VEGF, such as bevacizumab (e.g. AVASTIN™, Genentech, South San Francisco, Calif.), a recombinant humanized antibody to VEGF; integrin receptor antagonists and integrin antagonists, such as to αᵥβ₃, αᵥβ₅ and αᵥβ₆ integrins, and subtypes thereof, e.g. cilengitide (EMD 121974), or the anti-integrin antibodies, such as for example αᵥβ₃ specific humanized antibodies (e.g. VITAXIN®); factors such as IFN-alpha (U.S. Pat. Nos. 41,530,901, 4,503,035, and 5,231,176); angiostatin and plasminogen fragments (e.g. kringle 1-4, kringle 5, kringle 1-3 (O'Reilly, M. S. et al. (1994) Cell 79:315-328; Cao et al. (1996) J. Biol. Chem. 271: 29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1; Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-4701; suramin and suramin analogs; angiostatic steroids; bFGF antagonists; flk-1 and flt-1 antagonists; anti-angiogenesis agents such as MMP-2 (matrix-metalloproteinase 2) inhibitors and MMP-9 (matrix-metalloproteinase 9) inhibitors. Examples of useful matrix metalloproteinase inhibitors are described in International Patent Publication Nos. WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Signal transduction inhibitors include, for example: erbB2 receptor inhibitors, such as organic molecules, or antibodies that bind to the erbB2 receptor, for example, trastuzumab (e.g. HERCEPTIN®); inhibitors of other protein tyrosine-kinases, e.g. imitinib (e.g. GLEEVEC®); ras inhibitors; raf inhibitors; MEK inhibitors; mTOR inhibitors; cyclin dependent kinase inhibitors; protein kinase C inhibitors; and PDK-1 inhibitors (see Dancey, J. and Sausville, E. A. (2003) Nature Rev. Drug Discovery 2:92-313, for a description of several examples of such inhibitors, and their use in clinical trials for the treatment of cancer).

ErbB2 receptor inhibitors include, for example: ErbB2 receptor inhibitors, such as GW-282974 (Glaxo Wellcome plc), monoclonal antibodies such as AR-209 (Aronex Pharmaceuticals Inc. of The Woodlands, Tex., USA) and 2B-1 (Chiron), and erbB2 inhibitors such as those described in International Publication Nos. WO 98/02434, WO 99/35146, WO 99/35132, WO 98/02437, WO 97/13760, and WO 95/19970, and U.S. Pat. Nos. 5,587,458, 5,877,305, 6,465,449 and 6,541,481.

Antiproliferative agents include, for example: Inhibitors of the enzyme farnesyl protein transferase and inhibitors of the receptor tyrosine kinase PDGFR, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217. Antiproliferative agents also include inhibitors of the receptor tyrosine kinases IGF-1R and FGFR.

Examples of useful COX-II inhibitors include alecoxib (e.g. CELEBREX™), valdecoxib, and rofecoxib. Agents capable of enhancing antitumor immune responses include, for example: CTLA4 (cytotoxic lymphocyte antigen 4) antibodies (e.g. MDX-CTLA4), and other agents capable of blocking CTLA4. Specific CTLA4 antibodies that can be used in the present invention include those described in U.S. Pat. No. 6,682,736.

The use of the cytotoxic and other anticancer agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

The present invention further provides the methods described herein for treating tumors or tumor metastases in a patient comprising administering to the patient simultaneously or sequentially a therapeutically effective amount of an EGFR and/or IGF-1R kinase inhibitor, with in addition treatment with radiation or a radiopharmaceutical.

The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111. Where the EGFR or IGF-1R kinase inhibitor according to this invention is an antibody, it is also possible to label the antibody with such radioactive isotopes.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in International Patent Publication WO 99/60023.

For purposes of the present invention, “co-administration of” and “co-administering” an EGFR kinase inhibitor (or IGF-1R kinase inhibitor) and another agent (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. The other agent can be administered prior to, at the same time as, or subsequent to administration of the EGFR or IGF-1R kinase inhibitor, or in some combination thereof. Where the EGFR or IGF-1R kinase inhibitor is administered to the patient at repeated intervals, e.g., during a standard course of treatment, the mesenchymal-like cell kinase inhibitor can be administered prior to, at the same time as, or subsequent to, each administration of the EGFR or IGF-1R kinase inhibitor, or some combination thereof, or at different intervals in relation to the EGFR or IGF-1R kinase inhibitor treatment, or in a single dose prior to, at any time during, or subsequent to the course of treatment with the EGFR or IGF-1R kinase inhibitor.

The EGFR or IGF-1R kinase inhibitor will typically be administered to the patient in a dose regimen that provides for the most effective treatment of the cancer (from both efficacy and safety perspectives) for which the patient is being treated, as known in the art, and as disclosed, e.g. in International Patent Publication No. WO 01/34574. In conducting the treatment method of the present invention, the EGFR or IGF-1R kinase inhibitor can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the type of kinase inhibitor being used (for example, small molecule, antibody, RNAi, ribozyme or antisense construct), and the medical judgement of the prescribing physician as based, e.g., on the results of published clinical studies.

The amount of EGFR or IGF-1R kinase inhibitor administered, and the timing of kinase inhibitor administration, will depend on the type (species, gender, age, weight, etc.) and condition of the patient being treated, the severity of the disease or condition being treated, and on the route of administration. For example, small molecule kinase inhibitors can be administered to a patient in doses ranging from 0.001 to 100 mg/kg of body weight per day or per week in single or divided doses, or by continuous infusion (see for example, International Patent Publication No. WO 01/34574). In particular, erlotinib HCl can be administered to a patient in doses ranging from 5-200 mg per day, or 100-1600 mg per week, in single or divided doses, or by continuous infusion. A preferred dose is 150 mg/day. Antibody-based kinase inhibitors, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

The EGFR or IGF-1R kinase inhibitor can be administered either separately or together by the same or different routes, and in a wide variety of different dosage forms. For example, the EGFR or IGF-1R kinase inhibitor is preferably administered orally or parenterally. Where the EGFR kinase inhibitor is erlotinib HCl (TARCEVA®), oral administration is preferable. Both the EGFR or IGF-1R kinase inhibitors can be administered in single or multiple doses.

The EGFR or IGF-1R kinase inhibitor can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored.

The EGFR or IGF-1R kinase inhibitor can be combined together with various pharmaceutically acceptable inert carriers in the form of sprays, creams, salves, suppositories, jellies, gels, pastes, lotions, ointments, and the like. Administration of such dosage forms can be carried out in single or multiple doses. Carriers include solid diluents or fillers, sterile aqueous media, and various non-toxic organic solvents, etc.

All formulations comprising proteinaceous EGFR or IGF-1R kinase inhibitors, should be selected so as to avoid denaturation and/or degradation and loss of biological activity of the inhibitor.

Methods of preparing pharmaceutical compositions comprising an EGFR kinase inhibitor are known in the art, and are described, e.g. in International Patent Publication No. WO 01/34574. Methods of preparing pharmaceutical compositions comprising an IGF-1R kinase inhibitor are known in the art. In view of the teaching of the present invention, methods of preparing pharmaceutical compositions comprising both an EGFR and/or IGF-1R kinase kinase inhibitor will be apparent from the above-cited publications and from other known references, such as Remington's Pharmaceutical Sciences, Mack Publishing Company, Easton, Pa., 18^{th} edition (1990).

For oral administration of EGFR and/or IGF-1R kinase inhibitors tablets containing one or both of the active agents are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the EGFR or IGF-1R kinase inhibitors may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration of either or both of the active agents, solutions in either sesame or peanut oil or in aqueous propylene glycol may be employed, as well as sterile aqueous solutions comprising the active agent or a corresponding water-soluble salt thereof. Such sterile aqueous solutions are preferably suitably buffered, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art. Any parenteral formulation selected for administration of proteinaceous kinase inhibitors should be selected so as to avoid denaturation and loss of biological activity of the inhibitor.

Additionally, it is possible to topically administer either or both of the active agents, by way of, for example, creams, lotions, jellies, gels, pastes, ointments, salves and the like, in accordance with standard pharmaceutical practice. For example, a topical formulation comprising an EGFR or IGF-1R kinase inhibitor in about 0.1% (w/v) to about 5% (w/v) concentration can be prepared.

For veterinary purposes, the active agents can be administered separately or together to animals using any of the forms and by any of the routes described above. In a preferred embodiment, the EGFR or IGF-1R kinase inhibitor is administered in the form of a capsule, bolus, tablet, liquid drench, by injection or as an implant. As an alternative, the kinase inhibitor can be administered with the animal feedstuff, and for this purpose a concentrated feed additive or premix may be prepared for a normal animal feed. The kinase inhibitors can also be administered in the form of liquid drench, by injection, or as an implant. Such formulations are prepared in a conventional manner in accordance with standard veterinary practice.

As used herein, the term “EGFR kinase inhibitor” refers to any EGFR kinase inhibitor that is currently known in the art or that will be identified in the future, and includes any chemical entity that, upon administration to a patient, results in inhibition of a biological activity associated with activation of the EGF receptor in the patient, including any of the downstream biological effects otherwise resulting from the binding to EGFR of its natural ligand. Such EGFR kinase inhibitors include any agent that can block EGFR activation or any of the downstream biological effects of EGFR activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the EGF receptor, thereby making the receptor inaccessible to its natural ligand so that its normal biological activity is prevented or reduced. Alternatively, such an inhibitor can act by modulating the dimerization of EGFR polypeptides, or interaction of EGFR polypeptide with other proteins, or enhance ubiquitination and endocytotic degradation of EGFR. EGFR kinase inhibitors include but are not limited to low molecular weight inhibitors, antibodies, antibody fragments, peptide or RNA aptamers, antisense constructs, small inhibitory RNAs (i.e. RNA interference by dsRNA; RNAi), and ribozymes. In a preferred embodiment, the EGFR kinase inhibitor is a small organic molecule or an antibody that binds specifically to the human EGFR.

EGFR kinase inhibitors include, for example quinazoline EGFR kinase inhibitors, pyrido-pyrimidine EGFR kinase inhibitors, pyrimido-pyrimidine EGFR kinase inhibitors, pyrrolo-pyrimidine EGFR kinase inhibitors, pyrazolo-pyrimidine EGFR kinase inhibitors, phenylamino-pyrimidine EGFR kinase inhibitors, oxindole EGFR kinase inhibitors, indolocarbazole EGFR kinase inhibitors, phthalazine EGFR kinase inhibitors, isoflavone EGFR kinase inhibitors, quinalone EGFR kinase inhibitors, and tyrphostin EGFR kinase inhibitors, such as those described in the following patent publications, and all pharmaceutically acceptable salts and solvates of said EGFR kinase inhibitors: International Patent Publication Nos. WO 96/33980, WO 96/30347, WO 97/30034, WO 97/30044, WO 97/38994, WO 97/49688, WO 98/02434, WO 97/38983, WO 95/19774, WO 95/19970, WO 97/13771, WO 98/02437, WO 98/02438, WO 97/32881, WO 98/33798, WO 97/32880, WO 97/3288, WO 97/02266, WO 97/27199, WO 98/07726, WO 97/34895, WO 96/31510, WO 98/14449, WO 98/14450, WO 98/14451, WO 95/09847, WO 97/19065, WO 98/17662, WO 99/35146, WO 99/35132, WO 99/07701, and WO 92/20642; European Patent Application Nos. EP 520722, EP 566226, EP 787772, EP 837063, and EP 682027; U.S. Pat. Nos. 5,747,498, 5,789,427, 5,650,415, and 5,656,643; and German Patent Application No. DE 19629652. Additional non-limiting examples of low molecular weight EGFR kinase inhibitors include any of the EGFR kinase inhibitors described in Traxler, P., 1998, Exp. Opin. Ther. Patents 8(12):1599-1625.

Specific preferred examples of low molecular weight EGFR kinase inhibitors that can be used according to the present invention include [6,7-bis(2-methoxyethoxy)-4-quinazolin-4-yl]-(3-ethynylphenyl)amine (also known as OSI-774, erlotinib, or TARCEVA® (erlotinib HCl); OSI Pharmaceuticals/Genentech/Roche) (U.S. Pat. No. 5,747,498; International Patent Publication No. WO 01/34574, and Moyer, J. D. et al. (1997) Cancer Res. 57:4838-4848); CI-1033 (formerly known as PD183805; Pfizer) (Sherwood et al., 1999, Proc. Am. Assoc. Cancer Res. 40:723); PD-158780 (Pfizer); AG-1478 (University of California); CGP-59326 (Novartis); PKI-166 (Novartis); EKB-569 (Wyeth); GW-2016 (also known as GW-572016 or lapatinib ditosylate; GSK); and gefitinib (also known as ZD1839 or IRESSA™; Astrazeneca) (Woodburn et al., 1997, Proc. Am. Assoc. Cancer Res. 38:633). A particularly preferred low molecular weight EGFR kinase inhibitor that can be used according to the present invention is [6,7-bis(2-methoxyethoxy)-4-quinazolin-4-yl]-(3-ethynylphenyl) amine (i.e. erlotinib), its hydrochloride salt (i.e. erlotinib HCl, TARCEVA®), or other salt forms (e.g. erlotinib mesylate).

EGFR kinase inhibitors also include, for example multi-kinase inhibitors that have activity on EGFR kinase, i.e. inhibitors that inhibit EGFR kinase and one or more additional kinases. Examples of such compounds include the EGFR and HER2 inhibitor CI-1033 (formerly known as PD183805; Pfizer); the EGFR and HER2 inhibitor GW-2016 (also known as GW-572016 or lapatinib ditosylate; GSK); the EGFR and JAK 2/3 inhibitor AG490 (a tyrphostin); the EGFR and HER2 inhibitor ARRY-334543 (Array BioPharma); BIBW-2992, an irreversible dual EGFR/HER2 kinase inhibitor (Boehringer Ingelheim Corp.); the EGFR and HER2 inhibitor EKB-569 (Wyeth); the VEGF-R2 and EGFR inhibitor ZD6474 (also known as ZACTIMA™; AstraZeneca Pharmaceuticals), and the EGFR and HER2 inhibitor BMS-599626 (Bristol-Myers Squibb).

Antibody-based EGFR kinase inhibitors include any anti-EGFR antibody, including antibody fragments, that can partially or completely block EGFR activation by its natural ligand. Non-limiting examples of antibody-based EGFR kinase inhibitors include those described in Modjtahedi, H., et al., 1993, Br. J. Cancer 67:247-253; Teramoto, T., et al., 1996, Cancer 77:639-645; Goldstein et al., 1995, Clin. Cancer Res. 1:1311-1318; Huang, S. M., et al., 1999, Cancer Res. 15:59(8):1935-40; and Yang, X., et al., 1999, Cancer Res. 59:1236-1243. Thus, the EGFR kinase inhibitor can be the monoclonal antibody Mab E7.6.3 (Yang, X. D. et al. (1999) Cancer Res. 59:1236-43), or Mab C225 (ATCC Accession No. HB-8508), or an antibody, including antibody fragments, having the binding specificity thereof. Suitable monoclonal antibody EGFR kinase inhibitors include, but are not limited to, IMC-C225 (also known as cetuximab or ERBITUX™; Imclone Systems), ABX-EGF (Abgenix), EMD 72000 (Merck KgaA, Darmstadt), RH3 (York Medical Bioscience Inc.), and MDX-447 (Medarex/Merck KgaA).

EGFR kinase inhibitors for use in the present invention can alternatively be peptide or RNA aptamers. Such aptamers can for example interact with the extracellular or intracellular domains of EGFR to inhibit EGFR kinase activity in cells. An aptamer that interacts with the extracellular domain is preferred as it would not be necessary for such an aptamer to cross the plasma membrane of the target cell. An aptamer could also interact with the ligand for EGFR (e.g. EGF, TGF-α), such that its ability to activate EGFR is inhibited. Methods for selecting an appropriate aptamer are well known in the art. Such methods have been used to select both peptide and RNA aptamers that interact with and inhibit EGFR family members (e.g. see Buerger, C. et al. et al. (2003) J. Biol. Chem. 278:37610-37621; Chen, C-H. B. et al. (2003) Proc. Natl. Acad. Sci. 100:9226-9231; Buerger, C. and Groner, B. (2003) J. Cancer Res. Clin. Oncol. 129(12):669-675. Epub 2003 Sep 11.).

EGFR kinase inhibitors for use in the present invention can alternatively be based on antisense oligonucleotide constructs. Anti-sense oligonucleotides, including anti-sense RNA molecules and anti-sense DNA molecules, would act to directly block the translation of EGFR mRNA by binding thereto and thus preventing protein translation or increasing mRNA degradation, thus decreasing the level of EGFR kinase protein, and thus activity, in a cell. For example, antisense oligonucleotides of at least about 15 bases and complementary to unique regions of the mRNA transcript sequence encoding EGFR can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as EGFR kinase inhibitors for use in the present invention. EGFR gene expression can be reduced by contacting the tumor, subject or cell with a small double stranded RNA (dsRNA), or a vector or construct causing the production of a small double stranded RNA, such that expression of EGFR is specifically inhibited (i.e. RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494-498; Hannon, G. J. (2002) Nature 418:244-251; McManus, M. T. and Sharp, P. A. (2002) Nature Reviews Genetics 3:737-747; Bremmelkamp, T. R. et al. (2002) Science 296:550-553; U.S. Pat. Nos. 6,573,099 and 6,506,559; and International Patent Publication Nos. WO 01/36646, WO 99/32619, and WO 01/68836).

Ribozymes can also function as EGFR kinase inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of EGFR mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as EGFR kinase inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

As used herein, the term “IGF-1R kinase inhibitor” refers to any IGF-1R kinase inhibitor that is currently known in the art or that will be identified in the future, and includes any chemical entity that, upon administration to a patient, results in inhibition of a biological activity associated with activation of the IGF-1 receptor in the patient, including any of the downstream biological effects otherwise resulting from the binding to IGF-1R of its natural ligand. Such IGF-1R kinase inhibitors include any agent that can block IGF-1R activation or any of the downstream biological effects of IGF-1R activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the IGF-1 receptor, thereby making the receptor inaccessible to its natural ligand so that its normal biological activity is prevented or reduced. Alternatively, such an inhibitor can act by modulating the dimerization of IGF-1R polypeptides, or interaction of IGF-1R polypeptide with other proteins, or enhance ubiquitination and endocytotic degradation of IGF-1R. An IGF-1R kinase inhibitor can also act by reducing the amount of IGF-1 available to activate IGF-1R, by for example antagonizing the binding of IGF-1 to its receptor, by reducing the level of IGF-1, or by promoting the association of IGF-1 with proteins other than IGF-1R such as IGF binding proteins (e.g. IGFBP3). IGF-1R kinase inhibitors include but are not limited to low molecular weight inhibitors, antibodies, antibody fragments, antisense constructs, small inhibitory RNAs (i.e. RNA interference by dsRNA; RNAi), and ribozymes. In a preferred embodiment, the IGF-1R kinase inhibitor is a small organic molecule or an antibody that binds specifically to the human IGF-1R.

IGF-1R kinase inhibitors include, for example imidazopyrazine IGF-1R kinase inhibitors, azabicyclic amine inhibitors, quinazoline IGF-1R kinase inhibitors, pyrido-pyrimidine IGF-1R kinase inhibitors, pyrimido-pyrimidine IGF-1R kinase inhibitors, pyrrolo-pyrimidine IGF-1R kinase inhibitors, pyrazolo-pyrimidine IGF-1R kinase inhibitors, phenylamino-pyrimidine IGF-1R kinase inhibitors, oxindole IGF-1R kinase inhibitors, indolocarbazole IGF-1R kinase inhibitors, phthalazine IGF-1R kinase inhibitors, isoflavone IGF-1R kinase inhibitors, quinalone IGF-1R kinase inhibitors, and tyrphostin IGF-1R kinase inhibitors, and all pharmaceutically acceptable salts and solvates of such IGF-1R kinase inhibitors.

Examples of IGF-1R kinase inhibitors include those in International Patent Publication No. WO 05/097800, that describes azabicyclic amine derivatives, International Patent Publication No. WO 05/037836, that describes imidazopyrazine IGF-1R kinase inhibitors, International Patent Publication Nos. WO 03/018021 and WO 03/018022, that describe pyrimidines for treating IGF-1R related disorders, International Patent Publication Nos. WO 02/102804 and WO 02/102805, that describe cyclolignans and cyclolignans as IGF-1R inhibitors, International Patent Publication No. WO 02/092599, that describes pyrrolopyrimidines for the treatment of a disease which responds to an inhibition of the IGF-1R tyrosine kinase, International Patent Publication No. WO 01/72751, that describes pyrrolopyrimidines as tyrosine kinase inhibitors, and in International Patent Publication No. WO 00/71129, that describes pyrrolotriazine inhibitors of kinases, and in International Patent Publication No. WO 97/28161, that describes pyrrolo[2,3-d]pyrimidines and their use as tyrosine kinase inhibitors, Parrizas, et al., which describes tyrphostins with in vitro and in vivo IGF-1R inhibitory activity (Endocrinology, 138:1427-1433 (1997)), International Patent Publication No. WO 00/35455, that describes heteroaryl-aryl ureas as IGF-1R inhibitors, International Patent Publication No. WO 03/048133, that describes pyrimidine derivatives as modulators of IGF-1R, International Patent Publication No. WO 03/024967, WO 03/035614, WO 03/035615, WO 03/035616, and WO 03/035619, that describe chemical compounds with inhibitory effects towards kinase proteins, International Patent Publication No. WO 03/068265, that describes methods and compositions for treating hyperproliferative conditions, International Patent Publication No. WO 00/17203, that describes pyrrolopyrimidines as protein kinase inhibitors, Japanese Patent Publication No. JP 07/133,280, that describes a cephem compound, its production and antimicrobial composition, Albert, A. et al., Journal of the Chemical Society, 11: 1540-1547 (1970), which describes pteridine studies and pteridines unsubstituted in the 4-position, and A. Albert et al., Chem. Biol. Pteridines Proc. Int. Symp., 4th, 4: 1-5 (1969) which describes a synthesis of pteridines (unsubstituted in the 4-position) from pyrazines, via 3-4-dihydropteridines.

Additional, specific examples of IGF-1R kinase inhibitors that can be used according to the present invention include h7C10 (Centre de Recherche Pierre Fabre), an IGF-1 antagonist; EM-164 (ImmunoGen Inc.), an IGF-1R modulator; CP-751871 (Pfizer Inc.), an IGF-1 antagonist; lanreotide (Ipsen), an IGF-1 antagonist; IGF-1R oligonucleotides (Lynx Therapeutics Inc.); IGF-1 oligonucleotides (National Cancer Institute); IGF-1R protein-tyrosine kinase inhibitors in development by Novartis (e.g. NVP-AEW541, Garcia-Echeverria, C. et al. (2004) Cancer Cell 5:231-239; or NVP-ADW742, Mitsiades, C. S. et al. (2004) Cancer Cell 5:221-230); IGF-1R protein-tyrosine kinase inhibitors (Ontogen Corp); OSI-906 (OSI Pharmaceuticals); AG-1024 (Camirand, A. et al. (2005) Breast Cancer Research 7:R570—R579 (DOI 10.1186/bcr1028); Camirand, A. and Pollak, M. (2004) Brit. J. Cancer 90:1825-1829; Pfizer Inc.), an IGF-1 antagonist; the tyrphostins AG-538 and I-OMe-AG 538; BMS-536924, a small molecule inhibitor of IGF-1R; PNU-145156E (Pharmacia & Upjohn SpA), an IGF-1 antagonist; BMS 536924, a dual IGF-1R and IR kinase inhibitor (Bristol-Myers Squibb); AEW541 (Novartis); GSK621659A (Glaxo Smith-Kline); INSM-18 (Insmed); and XL-228 (Exelixis).

Antibody-based IGF-1R kinase inhibitors include any anti-IGF-1R antibody, including antibody fragments, that can partially or completely block IGF-1R activation by its natural ligand. Antibody-based IGF-1R kinase inhibitors also include any anti-IGF-1 antibody, including antibody fragments, that can partially or completely block IGF-1R activation. Non-limiting examples of antibody-based IGF-1R kinase inhibitors include those described in Larsson, O. et al (2005) Brit. J. Cancer 92:2097-2101 and Ibrahim, Y. H. and Yee, D. (2005) Clin. Cancer Res. 11:944s-950s; or being developed by Imclone (e.g. IMC-A12), or AMG-479, an anti-IGF-1R antibody (Amgen); R1507, an anti-IGF-1R antibody (Genmab/Roche); AVE-1642, an anti-IGF-1R antibody (Immunogen/Sanofi-Aventis); MK 0646 or h7C10, an anti-IGF-1R antibody (Merck); or antibodies being develop by Schering-Plough Research Institute (e.g. SCH 717454 or 19D12; or as described in US Patent Application Publication Nos. US 2005/0136063 A1 and US 2004/0018191 A1). The IGF-1R kinase inhibitor can be a monoclonal antibody, or an antibody, including antibody fragments, having the binding specificity thereof.

The term “pharmaceutically acceptable salts” refers to salts prepared from pharmaceutically acceptable non-toxic bases or acids. When a compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

When a compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

Pharmaceutical compositions useful in the present invention comprise an EGFR (and/or IGF-1R) kinase inhibitor (including pharmaceutically acceptable salts of each component thereof) as active ingredients, a pharmaceutically acceptable carrier and optionally other therapeutic ingredients or adjuvants. Other therapeutic agents may include those cytotoxic, chemotherapeutic or anti-cancer agents, or agents which enhance the effects of such agents, as listed above. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by an EGFR (and/or IGF-1R) kinase inhibitor (including pharmaceutically acceptable salts of each component thereof) of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, an EGFR (and/or IGF-1R) kinase inhibitor (including pharmaceutically acceptable salts of each component thereof) may also be administered by controlled release means and/or delivery devices. The combination compositions may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and an EGFR (and/or IGF-1R) kinase inhibitor (including pharmaceutically acceptable salts of each component thereof). An EGFR (and/or IGF-1R) kinase inhibitor (including pharmaceutically acceptable salts of each component thereof), can also be included in pharmaceutical compositions in combination with one or more other therapeutically active compounds. Other therapeutically active compounds may include those cytotoxic, chemotherapeutic or anti-cancer agents, or agents which enhance the effects of such agents, as listed above.

Thus in one embodiment of this invention, a pharmaceutical composition can comprise a combination of an EGFR (and/or IGF-1R) kinase inhibitor in combination with another anticancer agent, wherein said anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably contains from about 0.05 mg to about 5 g of the active ingredient.

For example, a formulation intended for the oral administration to humans may contain from about 0.5 mg to about 5 g of active agent, compounded with an appropriate and convenient amount of carrier material that may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions of the present invention can be in a form suitable for topical sue such as, for example, an aerosol, cream, ointment, lotion, dusting powder, or the like. Further, the compositions can be in a form suitable for use in transdermal devices. These formulations may be prepared, utilizing an EGFR (and/or IGF-1R) kinase inhibitor (including pharmaceutically acceptable salts of each component thereof) of this invention, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing an EGFR (and/or IGF-1R) kinase inhibitor (including pharmaceutically acceptable salts of each component thereof) may also be prepared in powder or liquid concentrate form.

Dosage levels for the compounds of the combination of this invention will be approximately as described herein, or as described in the art for these compounds. It is understood, however, that the specific dose level for any particular patient will depend upon a variety of factors including the age, body weight, general health, sex, diet, time of administration, route of administration, rate of excretion, drug combination and the severity of the particular disease undergoing therapy.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

Experimental Details:

Introduction

Epithelial and mesenchymal cells differ in various functional and phenotypic characteristics. Epithelial cells form layers of cells that are closely adjoined by specialized membrane structures including tight-junctions, adherens junctions, desmosomes and gap junctions. These cells demonstrate apical-basolateral polarization manifesting the distribution of adhesion molecules such as cadherins and certain integrins resulting in the organization of cell-cell junctions. Epithelial cells are motile and can move from their nearest neighbor while remaining within the epithelial layer. Mesenchymal cells, do not form an organized cell layer, and do not exhibit the same apical-basolateral organization and the polarization of cell surface molecules. They show only minimal contact with neighboring cells, and are not usually associated with the basal lamina. In culture mesenchymal cells have a spindle like morphology and tend to be highly motile, but this is not necessarily the case in vivo.

Epithelial cells can convert to mesenchymal cells by a process known as the epithelial-mesenchymal transistion or EMT. This term describes a series of events by which epithelial cells lose many of their epithelial characteristics and take on the properties of mesenchymal cells. This conversion requires complex changes in cell architecture and behavior. The transistion of the cellular characteristics involve a spectrum of inter and intracellular changes, not all of which are always observed during EMT. Therefore, EMT does not necessarily refer to a lineage switch. The spectrum of changes that occur during EMT is most likely determined by the integration of extracellular signals received. The reverse process, known as MET, has also been reported.

The hallmark of malignancy, invasion, consists in the translocation of tumor cells from the initial neoplastic focus into neighbouring host tissues, allowing tumor cells to penetrate vessel endothelium and enter the circulation forming distant metastasis. A histological pattern found at the periphery of carcinomas is the presence of individual malignant cells detached from the tumor mass remaining independent within the interstitial matrix of the stroma. While these cells are readily identified by the pathologist as invading malignant cells, their relationship with the compact-appearing portions of the tumor as well as the mechanism underlying the development of this pattern are not immediately evident at histological level. There is growing evidence suggesting that this change in tumor tissue architecture takes place through a peculiar phenotype modulation known as epithelial-mesenchymal transition (EMT). The essential features of EMT are the disruption of intercellular contacts and the enhancement of cell motility, thereby leading to the release of cells from the parent epithelial tissue. The resulting mesenchymal-like phenotype is suitable for migration, tumor invasion and dissemination allowing metastatic progression to proceed. Although the molecular bases of EMT have not been completely elucidated, several interconnected transduction pathways and a number of signalling molecules potentially involved have been identified. These include growth factors, receptor tyrosine kinases, Ras and other small GTPases, Src, beta-catenin and integrins. Most of these pathways converge on the down-regulation of the epithelial molecule E-cadherin, an event critical in tumor invasion and termed by some as a “master” programmer of EMT. The E-cadherin gene is somatically inactivated in many diffuse-type cancers such as lobular carcinoma of the breast and diffuse gastric carcinoma, in which neoplastic cells through the entire tumor mass have lost many of their epithelial characteristics and exhibit a highly invasive, EMT-derived histological pattern. E-cadherin down-modulation is also seen in solid, non-diffuse-type cancers at the tumor-stroma boundary where singly invading, EMT-derived tumor cells are observed in histological sections. In this latter scenario, E-cadherin loss and EMT could be transient, reversible processes possibly regulated by the tumor microenvironment. Neoplastic cells that have undergone EMT during invasion appear to regain E-cadherin expression. Since the molecules involved in EMT represent potential targets for pharmacological agents, these findings open new avenues for the identification of primary tumors and metastatic spread using molecular imaging modalities, leading to more appropriate and efficacious treatment regimens for such malignancies.

Materials and Methods

E-Cadherin antibody was obtained from Santa Cruz Biotechnology (Santa Cruz, Calif.; product No. sc-7870 L) at a concentration of 2.0 mg/mL in phosphate buffered saline (PBS), and free of gelatin and sodium azide. This antibody is a rabbit polyclonal IgG that can bind to either human or mouse E-cadherin. It was raised against amino acids 600-707 mapping within the extracellular domain of E-cadherin of human origin (Tanihara, et al. 1994. Cell Adhes. Commun. 2: 15-26).

An AF680 Labeling Kit was obtained from Invitrogen (Product No. S30045; Carlsbad, Calif.).

“H358 cells” as used herein, refer to cells of the cell line NCI-H358™ [a.k.a. H-358; H358] available from the American Tissue Culture Collection (ATCC) as CRL-5807, derived form human lung bronchiole or alveolus and showing morphology of both bronchioalveolar carcinoma and non-small cell lung adenocarcinoma. The H358 cell line, BxPC3 cells, and MDA-MB-231 cells were all obtained from ATCC, and were grown in media as prescribed by the ATCC, containing 10% FCS.

The H358 Tet-ON Zeb LV 198 animal model (i.e. nude mouse xenograft) was generated in-house at OSI-Boulder using standard techniques, using TET-inducible Zeb-1 H358 cells prepared essentially as described below.

Generation of TET-Responsive Cell Lines

H358 cells were seeded in 90 mm TC dish at a density to ensure 80% confluence after 24 hours of growth. The plasmids ptTS and prTA were transected into the cells at a 10:1 ratio using Fugene HD transfection reagent (Fugene). After 4 hours the media was removed and replaced with normal growth media and the cells allowed to grow for a further 48 hours. The cells were then split at different ratios (1:25, 1:50 and 1:100) into 150 mm TC dishes and allowed to grow for 24 hours. Drug (blasticidin, 100 μg/ml) was then added to the media and cell colonies selected over a 3-4 week period with the Bsd concentration being gradually reduced to 10 ug/ml. Colonies arising from single cells were picked from the plates using cell colony filters and expanded. The clones were screened for those which showed a tightly regulated inducible expression of a transiently introduced TET-responsive luciferase expression plasmid, as assayed by a luciferase assay (Steady Glo, Promega). A >10 fold induction of luciferase expression in response to doxycycline expression was considered adequate for further cell line construction.

Generation of TET-Inducible Gene Cell Lines

Plasmids containing full length cDNAs encoding Snail, Zeb1, or TGFbeta (constitutively active) (Snail mRNA sequence, Genbank NM_{—}005985, product of GeneID: 6615; Zeb1 mRNA sequence, Genbank NM_{—}030751, product of GeneID: 6935; TGFbeta sequence encoding constitutively active Ser223/S225 human TGF-beta-1 (i.e. Genbank NP_{—}000651 (product of GeneID: 7040), with cysteines 223 and 225 mutated to serine) under the control of a Tet-regulated promoter (pTRE2; Invitrogen) were constructed using standard methods. The TET-ON cell lines were plated and transfected with a pTRE2-Snail, pTRE2-Zeb1, or pTRE2-TGFbeta plasmid as described above. Once plated into 150 mm dishes the single cells were selected using puromycin (0.5 μg/mL). Colonies were selected over a 3-4 weeks period with puromycin concentration being reduced to a final concentration of 0.1 μg/ml. Colonies were picked using colony filters and screened for TET-dependent expression of the target gene by western blot analysis. In some cases multiple cDNAs were cotransfected into a given cell line. These methods enable the generation of cell lines which undergo EMT in response to tetracycline or analogs thereof, driven by the cDNAs listed above.

Synthesis of the AF680 E-Cadherin NIR Probe: The E-cadherin antibody was labeled with the AF680 dye by incubation of the antibody (1.0 mg) with the NHS activated ester, sodium bicarbonate (504) and Regulator solution (10 μL) for 60 minutes at room temperature according to the manufacture's labeling instructions (SAIVI Rapid Antibody Labeling Kit, Invitrogen), and protected from light. The crude reaction mixture was then placed on a size exclusion column and the dye-conjugate eluted using PBS as the eluant. Unreacted dye remained on the column. Following chromatographic separation the Degree of Labeling (DoL) of the dye-conjugate was determined spectrophotometrically by determining the peak absorbances of the dye-conjugate (A₂₈₀) and the unreacted dye (A₆₇₉). The DoL was calculated from the formula provided by Invitrogen. Typically the DoL (mole fluorophore:mole antibody) obtained for the E-Cadherin conjugates ranged from 1.20-2.0.

Synthesis of the NS (non-specific) NIR Probe: This probe was synthesized using the same procedure as for the E-Cadherin NIR probe. The Degree of Labeling (DoL) for this antibody NIR probe was determined to be 1.66. The NS antibody used was a rabbit anti-rat IgG (H+L) from Jackson ImmunoResearch Laboratories (West Grove, Pa.), Cat. #312-005-003.

Animal Models: H358 tumor NSCLC cells were maintained in RPMI media supplemented with 10% FCS and 1% sodium pyruvate. Female CD-1 athymic nu/nu mice (i.e. “nude mice”; Charles Rivers Laboratories) were inoculated subcutaneously in the flank region with 1.0×10⁷ cells in a 50:50 mixture (100 μL) of PBS:Matrigel. Palpable tumors are evident in the mice at approximately seven days post inoculation of cells. This cell line is positive for cell surface E-cadherin expression.

MDA-MB-231 cells were grown in IMDM media supplemented with 10% FCS. Female CD-1 athymic nu/nu mice (Charles Rivers Laboratories) were inoculated subcutaneously in the flank region with 1.0×10⁷ cells (100 μL) of PBS. Palpable tumors are evident in the mice at approximately fourteen days post inoculation of cells. This cell line is largely negative for the expression of cell surface E-cadherin.

BxPC-3 (human pancreatic) cells were grown in RPMI media supplemented with 10% FCS. Female CD-1 athymic nu/nu mice (Charles Rivers) were inoculated subcutaneously in the upper thoracic region with 1.0×10⁷ cells (100 μL) of PBS. Palpable tumors are evident in the mice at approximately fourteen days post inoculation of cells. This cell line is positive for cell surface E-cadherin.

Molecular Imaging: The tumor bearing mice are then injected with 10-100 μg of E-cadherin NIR probe via the lateral tail vein. Imaging of the mice is performed using an IVIS® Spectrum (Caliper Life Sciences, Hopkinton, Mass., USA). Images are acquired beginning at 75 minutes post injection of the NIR probe through 240 hours (10 days). Acquisition parameters for in vivo imaging are: excitation wavelength at 640 nm and emission wavelength collected at 720 nm. Typically acquisition time is approximately 2-15 seconds per animal. The animals are place in a prone or lateral position depending on tumor type within the imaging chamber and imaged using the aforementioned excitation and emission wavelengths. Resulting images are stored for later image analysis using the Living Image 3.0 Software package.

Results and Discussion

Experiment I: AF680 E-Cadherin NIR Probe Localization Study in vivo.

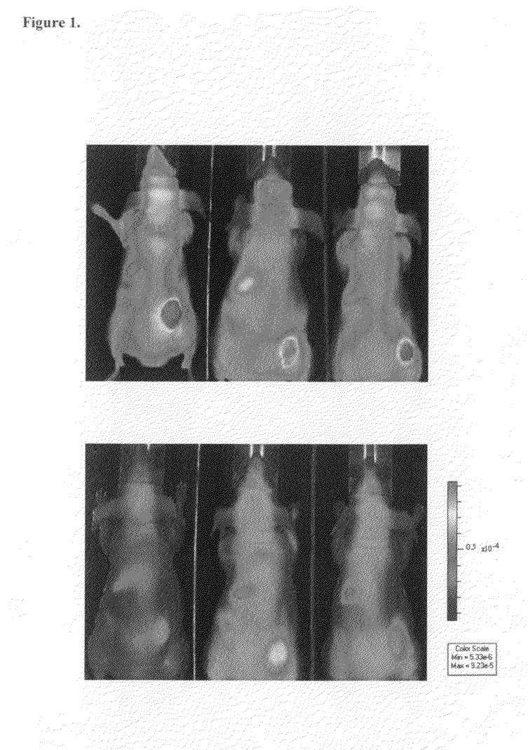

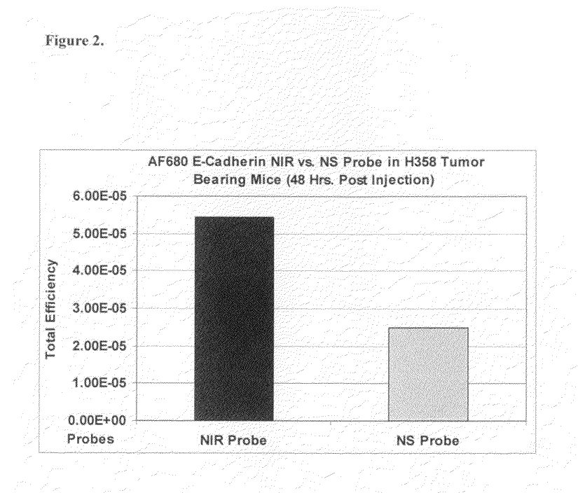

H358 tumor bearing nu/nu mice were injected with 40 μg of E-cadherin NIR probe (FIG. 1-Top Panel) and with the same mass of AF680 labeled non-specific IgG irrelevant antibody. (FIG. 1-Bottom Panel). Images were acquired at 48 hours post injection of the respective NIR probes. Excitation was at 640 nm and emission collected at 720 nm. H358 tumors are clearly visible in the right flank area in the animals receiving the AF680 E-cadherin NIR probe, while the animals receiving the non-specific IgG probe show barely visible tumor masses. Quantification of the AF680 E-cadherin NIR probe localization vs. non-specific IgG localization by ROI (Region of Interest) determination demonstrates a 2.5 fold increase of the E-cadherin probe in the H358 tumors vs. the non-specific probe (FIG. 2). A similar result was obtained using an epithelial mouse tumor cell line (i.e. the 4T1 mouse mammary cell line, ATCC® Number CRL-2539™) instead of human H358 cells.

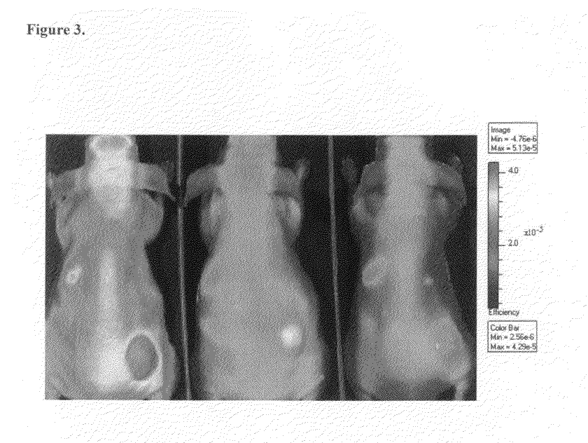

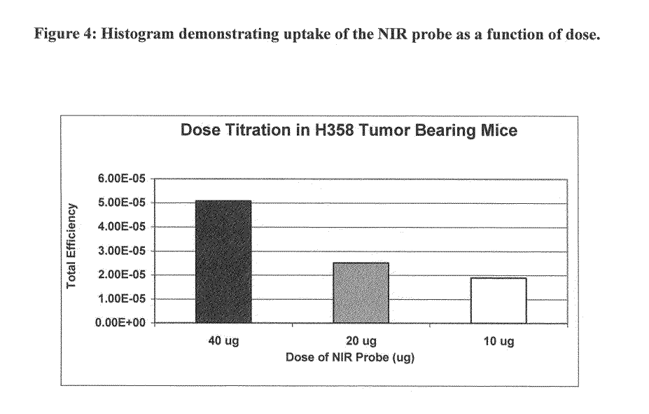

Titration of the AF680 E-cadherin NIR probe at 40 μg (A), 20 μg (B) and 10 μg (C) per tumor bearing mouse is demonstrated in FIG. 3. Image shows a progressive decrease in signal intensity of the probe as a function of injected dose. Imaging parameters were: Excitation wavelength 640 nm and excitation wavelength at 720 nm. The accompanying histogram in FIG. 4 demonstrates quantitatively the uptake of NIR probe per tumor as a function of dose.

Experiment II: PK Study of AF680 E-Cadherin NIR Probe in Tumor Bearing Mice

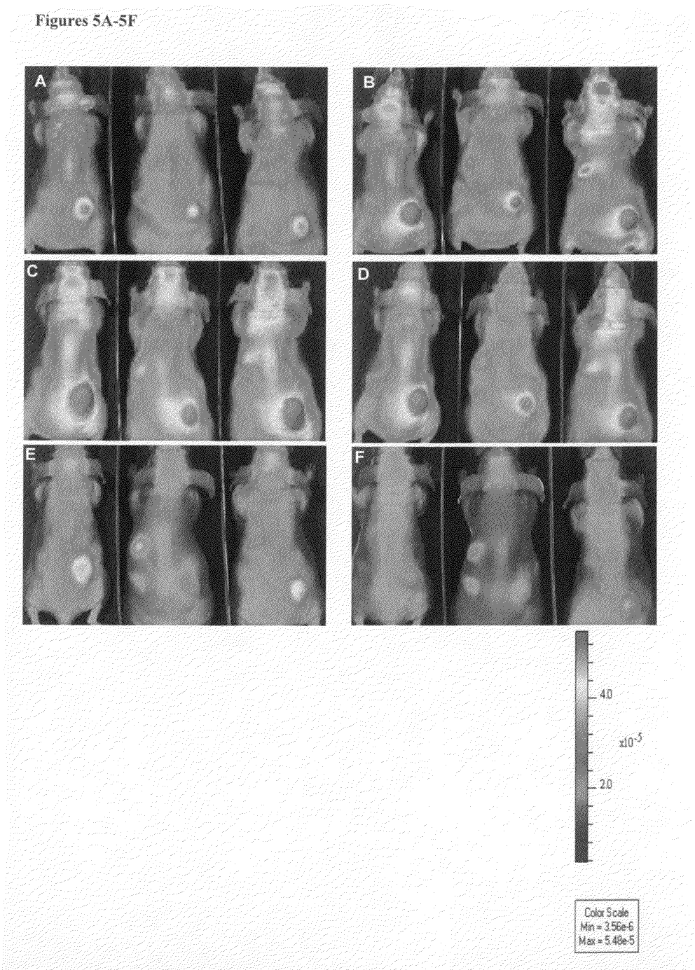

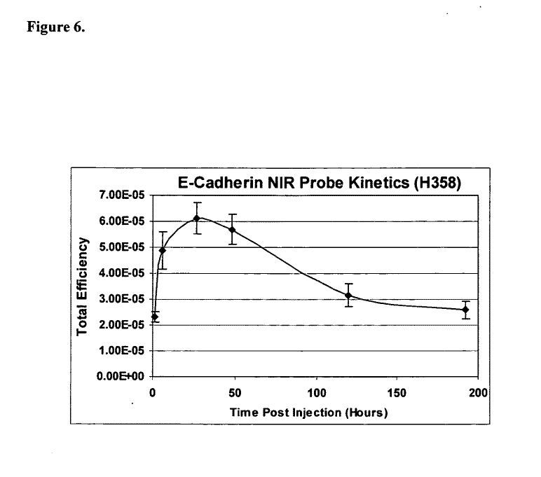

A pharmacokinetic (PK) study was conducted in H358 tumor bearing mice injected with 20 μg of E-cadherin NIR probe (n=3) and serially imaged at 75 minutes, 6 hours, 27 hours, 48 hours, 120 hours and 192 hours post injection of the NIR probe (Panels 5A-5F). Images show increase in signal intensity through 27 hours with a decline in signal intensity thereafter (FIG. 5). Accompanying graph depicts absorption and clearance kinetics of the probe over time from these animals (FIG. 6).

Experiment III: Demonstration of Probe Specificity Study In Vivo.

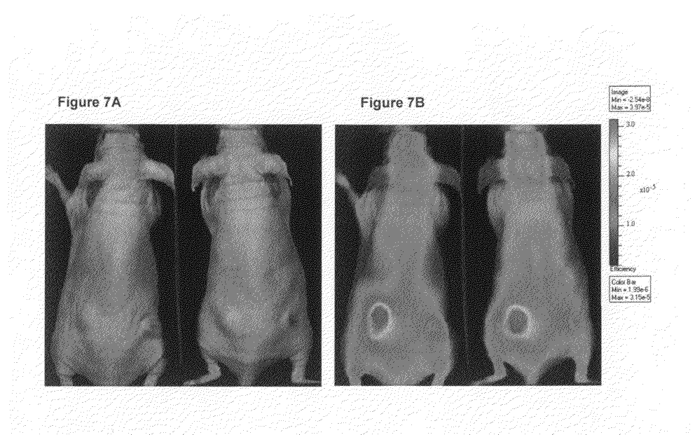

Female CD-1 nu/nu athymic mice were inoculated bilaterally with H358 in the left flank area, and with MDA-MB-231 cells in the right flank area (FIG. 7A-Photographic Image). The H358 xenografts are positive for cell surface E-cadherin expression while the MDA-MB-213 xenografts are negative for cell surface E-cadherin expression. When the two tumor masses reached equivalent mass, the animals were injected with 20 □g of the AF680 E-Cadherin NIR probe via the lateral tail vein. The mice were imaged at 48 hours post injection of the probe. FIG. 7B demonstrates the specificity of the NIR probe for the E-cadherin expressing tumor (H358-left flank) compared to the MDA-MB-231 (right flank) E-cadherin negative tumor.

Experiment IV: Localization and Quantification of Uptake In Vivo.

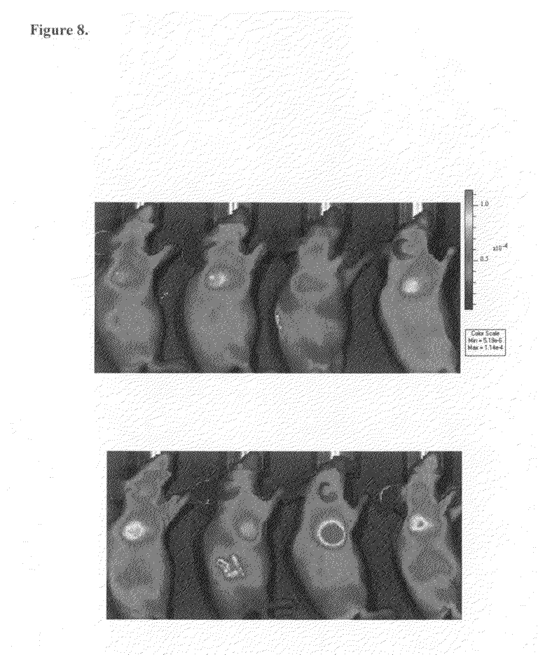

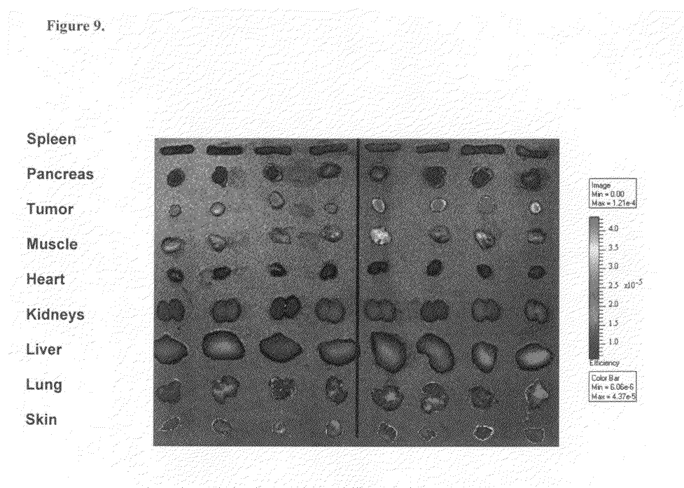

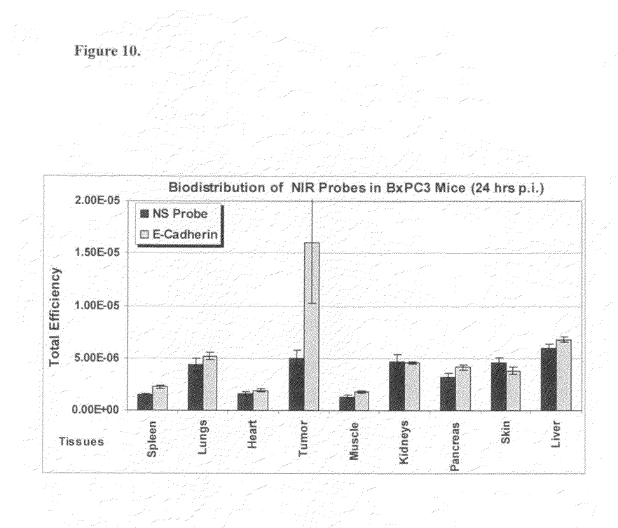

BxPC-3 tumor bearing nu/nu mice were injected with 40 μg of E-cadherin NIR probe and with the same mass of AF680 labeled non-specific IgG irrelevant antibody. At 24 hours post injection of the probes the mice were imaged, and selected tissues were then excised from the animals and images were acquired. Excitation was at 640 nm and emission collected at 720 nm. BxPC3 tumors from the mice injected with the AF680 E-cadherin NIR probe are clearly visible, in vivo (FIG. 8) and ex vivo (FIG. 9) compared to the tumors receiving the AF680 non-specific IgG NIR probe. Quantification of the AF680 E-Cadherin NIR probe localization vs. non-specific IgG localization by ROI determination demonstrates a greater than three fold increase of the E-cadherin probe in the tumors versus the non-specific probe in this tumor type (FIG. 10).

Experiment V: Imaging of EMT In Vivo.

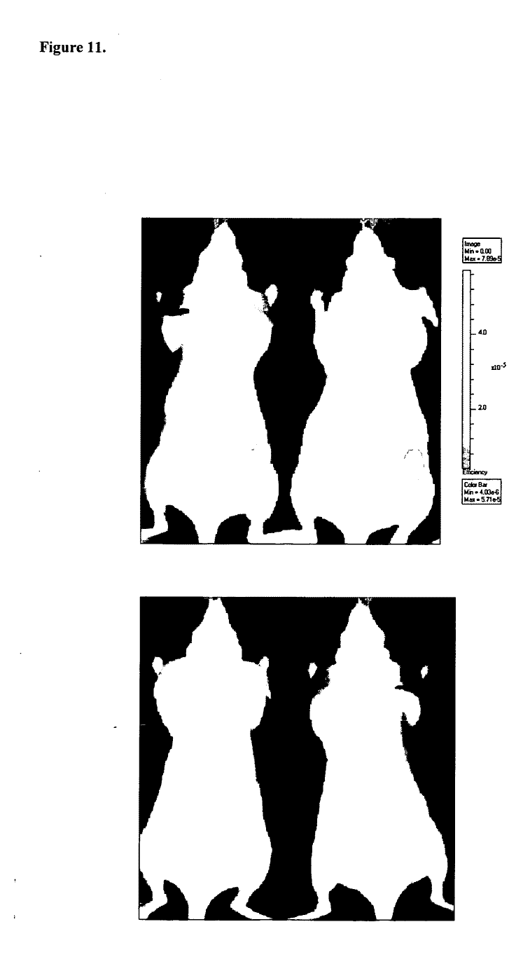

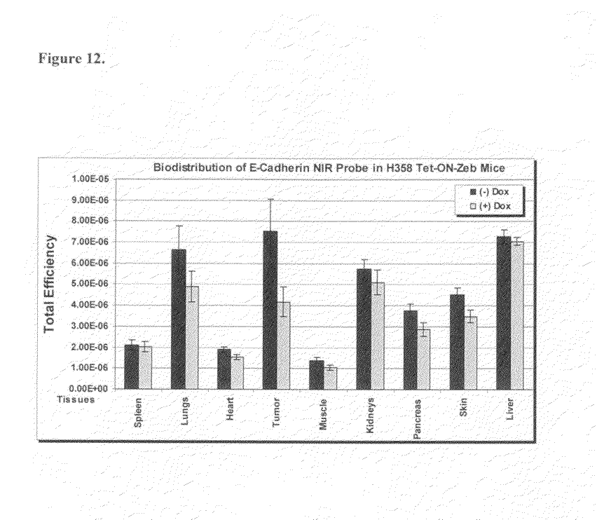

Female CD-1 athymic nu/nu mice bearing H358 Tet-ON Zeb LV-195 xenografts were separated in two groups (n=4). The positive control group received normal drinking water for 7 days, while the negative control group received water containing 0.5 mg/mL doxycycline. The doxycycline induces the gene Zeb which represses E-cadherin expression in vivo. After 7 days of doxycycline treatment all mice were in injected with 20 μg of the AF680 E-cadherin NIR probe and imaged 48 hours post injection (FIG. 11). From these mice selected tissues were removed and imaged ex vivo. Uptake of the NIR probe in the positive control group (no doxycycline) was approximately 45% greater than the group receiving doxycycline treatment demonstrating that changes in E-cadherin expression in vivo can be imaged and quantitated using this E-cadherin NIR probe as the biomarker of in vivo EMT changes (FIG. 12).

### ABBREVIATIONS

NIR, near infrared; AF680, Alexa Fluor 680 dye; EGF, epidermal growth factor; EGFR, epidermal growth factor receptor; EMT, epithelial-to-mesenchymal transition; MET, mesenchymal-to-epithelial transition; NSCL, non-small cell lung; NSCLC, non-small cell lung cancer; HNSCC, head and neck squamous cell carcinoma; CRC, colorectal cancer; MBC, metastatic breast cancer; Brk, Breast tumor kinase (also known as protein tyrosine kinase 6 (PTK6)); FCS, fetal calf serum; LC, liquid chromatography; MS, mass spectrometry; ROI, Region of Interest; IGF-1, insulin-like growth factor-1; TGFα, transforming growth factor alpha; HB-EGF, heparin-binding epidermal growth factor; IC₅₀, half maximal inhibitory concentration; pY, phosphotyrosine; wt, wild-type; PI3K, phosphatidyl inositol-3 kinase; GAPDH, glyceraldehyde 3-phosphate dehydrogenase; MAPK, mitogen-activated protein kinase; PDK-1,3-Phosphoinositide-Dependent Protein Kinase 1; Akt, also known as protein kinase B, is the cellular homologue of the viral oncogene v-Akt; mTOR, mammalian target of rapamycin; 4EBP1, eukaryotic translation initiation factor-4E (mRNA cap-binding protein) Binding Protein-1, also known as PHAS-I; p70S6K, 70 kDa ribosomal protein-S6 kinase; eIF4E, eukaryotic translation initiation factor-4E (mRNA cap-binding protein); Raf, protein kinase product of Raf oncogene; MEK, ERK kinase, also known as mitogen-activated protein kinase kinase; ERK, Extracellular signal-regulated protein kinase, also known as mitogen-activated protein kinase; PTEN, “Phosphatase and Tensin homologue deleted on chromosome 10”, a phosphatidylinositol phosphate phosphatase; pPROTEIN, phospho-PROTEIN, “PROTEIN” can be any protein that can be phosphorylated, e.g. EGFR, ERK, S6 etc; PBS, Phosphate-buffered saline; TGI, tumor growth inhibition; WFI, Water for Injection; SDS, sodium dodecyl sulfate; ErbB2, “v-erb-b2 erythroblastic leukemia viral oncogene homolog 2”, also known as HER-2; ErbB3, “v-erb-b2 erythroblastic leukemia viral oncogene homolog 3”, also known as HER-3; ErbB4, “v-erb-b2 erythroblastic leukemia viral oncogene homolog 4”, also known as HER-4; FGFR, Fibroblast Growth Factor Receptor; DMSO, dimethyl sulfoxide; HGF, hepatocyte growth factor; Wnt, wingless-type MMTV integration site family, member 1; IL-1, interleukin 1; HB-EGF, heparin-binding EGF-like growth factor; MSP, macrophage-stimulating protein; Wnt5a, wingless-type MMTV integration site family, member 5a; Shh, sonic hedgehog; TNF-alpha, transforming growth factor-α.

### INCORPORATION BY REFERENCE

All patents, published patent applications and other references disclosed herein are hereby expressly incorporated herein by reference.

### EQUIVALENTS

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A method of determining in situ the EMT status of the cells of a tumor in a patient, comprising:
(a) providing an EMT-status-detecting conjugate comprising an antibody that binds to an EMT-status biomarker, and a reporter molecule that produces a detectable signal;
(b) introducing said conjugate into a patient with a tumor, such that the conjugate contacts the EMT-status biomarker of the cells of the tumor;
(c) employing a means for detection of the signal from the conjugate at the tumor site; and
(d) using a means for image analysis to localize and quantify the signal from the conjugate at the tumor site,
a positive signal indicating the presence of epithelial tumor cells if the EMT-status biomarker is an epithelial biomarker, and mesenchymal tumor cells if the EMT-status biomarker is a mesenchymal biomarker.

2. The method of claim 1, wherein the EMT-status biomarker is E-cadherin, Erb-B3, S100-P, S100-A6, TACD1, CD98, MUC1, ZO-1 EFNB2, FLRT3, SPARC or CD44.

3. The method of claim 1, wherein the reporter molecule comprises an NIR dye.

4. The method of claim 1, wherein the reporter molecule comprises a radionuclide.

5. A method of identifying an agent that inhibits the cells of a tumor in a patient from undergoing an epithelial to mesenchymal transition, wherein the patient is an animal model of cancer comprising:
(a) introducing a test agent that into said patient such that the agent contacts the cells of the tumor;
(b) contacting the patient with an agent capable of inducing the cells of the tumor to undergo EMT;
(c) providing an EMT-status-detecting conjugate comprising an antibody that binds to an EMT-status biomarker, and a reporter molecule that produces a detectable signal;
(d) introducing said conjugate into the patient, such that the conjugate contacts the EMT-status biomarker of the cells of the tumor;
(e) employing a means for detection of the signal from the conjugate at the tumor site;
(f) using a means for image analysis to localize and quantify the signal from the conjugate at the tumor site,
a positive signal indicating the presence of epithelial tumor cells if the EMT-status biomarker is an epithelial biomarker, and mesenchymal tumor cells if the EMT-status biomarker is a mesenchymal biomarker, and
(g) comparing the signal in (f) with that produced in a similarly treated patient but that was not treated with the test agent as in (a), thus identifying whether said test agent is an agent that can inhibit tumor cells from undergoing an epithelial to mesenchymal transition.

6. The method of claim 5, wherein the EMT-status biomarker is E-cadherin, Erb-B3, S100-P, S100-A6, TACD1, CD98, MUC1, ZO-1 EFNB2, FLRT3, SPARC or CD44.

7. The method of claim 5, wherein the reporter molecule comprises an NIR dye.

8. The method of claim 5, wherein the reporter molecule comprises a radionuclide.

9. A method of identifying an agent that inhibits the cells of a tumor in a patient from undergoing an epithelial to mesenchymal transition, comprising:
(a) introducing a test agent that into said patient such that the agent contacts the cells of the tumor;
(b) providing an EMT-status-detecting conjugate comprising an antibody that binds to an EMT-status biomarker, and a reporter molecule that produces a detectable signal;
(c) introducing said conjugate into the patient, such that the conjugate contacts the EMT-status biomarker of the cells of the tumor;
(d) employing a means for detection of the signal from the conjugate at the tumor site;
(e) using a means for image analysis to localize and quantify the signal from the conjugate at the tumor site,
a positive signal indicating the presence of epithelial tumor cells if the EMT-status biomarker is an epithelial biomarker, and mesenchymal tumor cells if the EMT-status biomarker is a mesenchymal biomarker, and
(f) comparing the signal in (e) with that produced in a similarly treated patient but that was not treated with the test agent as in (a), thus identifying whether said test agent is an agent that can inhibit tumor cells from undergoing an epithelial to mesenchymal transition.

10. The method of claims 9, wherein the EMT-status biomarker is E-cadherin, Erb-B3, S100-P, S100-A6, TACD1, CD98, MUC1, ZO-1 EFNB2, FLRT3, SPARC or CD44.

11. The method of claim 9, wherein the reporter molecule comprises an NIR dye.

12. The method of claim 9, wherein the reporter molecule comprises a radionuclide.

13. A method for treating tumors in a patient with cancer, comprising:
(a) assessing the EMT status of the tumor cells by the method of claim 1, and
(b) administering to said patient a therapeutically effective amount of an EGFR-kinase inhibitor.

14. The method of claim 13, wherein the EGFR-kinase inhibitor comprises erlotinib.

15. A method for treating tumors in a patient with cancer, comprising:
(a) assessing the EMT status of the tumor cells by the method of claim 1, and
(b) administering to said patient a therapeutically effective amount of an IGF-1R-kinase inhibitor.

16. The method of claim 15, wherein the IGF-1R-kinase inhibitor comprises OSI-906.

17. A composition comprising an EMT-status-detecting conjugate comprising an antibody that binds to an extracellular domain of an EMT-status biomarker and a reporter molecule that produces a detectable signal, for use in a method of determining in situ the EMT status of the cells of a tumor in a patient.

18. A method of determining in situ the EMT status of the cells of a tumor in a patient, comprising:
(a) providing an EMT-status-detecting conjugate comprising an AFFIBODY® molecule that binds to an EMT-status biomarker, and a reporter molecule that produces a detectable signal;
(b) introducing said conjugate into a patient with a tumor, such that the conjugate contacts the EMT-status biomarker of the cells of the tumor;
(c) employing a means for detection of the signal from the conjugate at the tumor site; and
(d) using a means for image analysis to localize and quantify the signal from the conjugate at the tumor site,
a positive signal indicating the presence of epithelial tumor cells if the EMT-status biomarker is an epithelial biomarker, and mesenchymal tumor cells if the EMT-status biomarker is a mesenchymal biomarker.

19. The method of claim 18, wherein the EMT-status biomarker is E-cadherin, Erb-B3, S100-P, S100-A6, TACD1, CD98, MUC1, ZO-1 EFNB2, FLRT3, SPARC or CD44.

20. The method of claim 18, wherein the reporter molecule comprises an NIR dye.

21. The method of claim 18, wherein the reporter molecule comprises a radionuclide.

22. A method of identifying an agent that inhibits the cells of a tumor in a patient from undergoing an epithelial to mesenchymal transition, wherein the patient is an animal model of cancer comprising:
(a) introducing a test agent that into said patient such that the agent contacts the cells of the tumor;
(b) contacting the patient with an agent capable of inducing the cells of the tumor to undergo EMT;
(c) providing an EMT-status-detecting conjugate comprising an AFFIBODY® molecule that binds to an EMT-status biomarker, and a reporter molecule that produces a detectable signal;
(d) introducing said conjugate into the patient, such that the conjugate contacts the EMT-status biomarker of the cells of the tumor;
(e) employing a means for detection of the signal from the conjugate at the tumor site;
(f) using a means for image analysis to localize and quantify the signal from the conjugate at the tumor site,
a positive signal indicating the presence of epithelial tumor cells if the EMT-status biomarker is an epithelial biomarker, and mesenchymal tumor cells if the EMT-status biomarker is a mesenchymal biomarker, and
(g) comparing the signal in (f) with that produced in a similarly treated patient but that was not treated with the test agent as in (a), thus identifying whether said test agent is an agent that can inhibit tumor cells from undergoing an epithelial to mesenchymal transition.

23. The method of claim 22, wherein the EMT-status biomarker is E-cadherin, Erb-B3, S100-P, S100-A6, TACD1, CD98, MUC1, ZO-1 EFNB2, FLRT3, SPARC or CD44.

24. The method of claim 22, wherein the reporter molecule comprises an NIR dye.

25. The method of claim 22, wherein the reporter molecule comprises a radionuclide.

26. A method of identifying an agent that inhibits the cells of a tumor in a patient from undergoing an epithelial to mesenchymal transition, comprising:
(a) introducing a test agent that into said patient such that the agent contacts the cells of the tumor;
(b) providing an EMT-status-detecting conjugate comprising an AFFIBODY® molecule that binds to an EMT-status biomarker, and a reporter molecule that produces a detectable signal;
(c) introducing said conjugate into the patient, such that the conjugate contacts the EMT-status biomarker of the cells of the tumor;
(d) employing a means for detection of the signal from the conjugate at the tumor site;
(e) using a means for image analysis to localize and quantify the signal from the conjugate at the tumor site,
a positive signal indicating the presence of epithelial tumor cells if the EMT-status biomarker is an epithelial biomarker, and mesenchymal tumor cells if the EMT-status biomarker is a mesenchymal biomarker, and
(f) comparing the signal in (e) with that produced in a similarly treated patient but that was not treated with the test agent as in (a), thus identifying whether said test agent is an agent that can inhibit tumor cells from undergoing an epithelial to mesenchymal transition.

27. A method for treating tumors in a patient with cancer, comprising:
(a) assessing the EMT status of the tumor cells by the method of claim 15, and
(b) administering to said patient a therapeutically effective amount of an EGFR-kinase inhibitor.

28. The method of claim 27, wherein the EGFR-kinase inhibitor comprises erlotinib.

29. A method for treating tumors in a patient with cancer, comprising:
(a) assessing the EMT status of the tumor cells by the method of claim 15, and
(b) administering to said patient a therapeutically effective amount of an IGF-1R-kinase inhibitor.

30. The method of claim 29, wherein the IGF-1R-kinase inhibitor comprises OSI-906.

31. A composition comprising an EMT-status-detecting conjugate comprising an AFFIBODY® molecule that binds to an extracellular domain of an EMT-status biomarker and a reporter molecule that produces a detectable signal, for use in a method of determining in situ the EMT status of the cells of a tumor in a patient.
